(19) United States
(12) Patent Application Publication
(10) Pub. No.: US 2015/0079624 A1
(43) Pub. Date: Mar. 19, 2015

(54) PORIFERA-BASED THERAPEUTIC COMPOSITIONS FOR TREATING AND PREVENTING SKIN DISEASES

(71) Applicant: Maria Villani, Newport Beach, CA (US)

(72) Inventor: Maria Villani, Newport Beach, CA (US)

(21) Appl. No.: 14/553,694

(22) Filed: Nov. 25, 2014

Related U.S. Application Data
(63) Continuation of application No. 13/777,815, filed on Feb. 26, 2013, now abandoned.
Application No. 13/777,815 is a continuation-in-part of application No. 12/543,843, filed on Aug. 19, 2009, now Pat. No. 8,383,100.
Application No. 12/543,843 is a continuation of application No. 10/659,451, filed on Sep. 9, 2003, now Pat. No. 7,604,821.
Application No. 10/659,451 is a continuation-in-part of application No. 10/186,996, filed on Jul. 1, 2002, now abandoned.

(51) Int. Cl. A61K 35/56; G01N 30/06; G01N 33/50; G01N 33/483; A61K 9/14; B02C 23/12

(52) U.S. Cl. CPC A61K 35/56; A61K 9/14; B02C 23/12; G01N 33/5044; G01N 33/4833; G01N 30/06
USPC 435/29; 241/24.1; 73/61.52

(57) ABSTRACT

Methods and protocols of harvesting and processing of Porifera species, specifically sponges, and more specifically fresh water species of Genus Spongilla; testing and storing of the processed Spongilla powder; and manufacturing and packaging of Spongilla-based therapeutic compositions for treating and preventing skin diseases are disclosed. Treatable skin conditions and diseases include without limitation acne vulgaris, rosacea, seborrheic dermatitis, psoriasis, photo-aging and actinic keratosis, acne vulgaris, psoriasis, photo-aging, wounds, scars, and eczema (atopic dermatitis).

### RELATED APPLICATIONS

The present application is a continuation of U.S. patent application Ser. No. 13/777,815, filed Feb. 26, 2013, which is a continuation-in-part of U.S. patent application Ser. No. 12/543,843 filed Aug. 19, 2009, now U.S. Pat. No. 8,383,100, which is a continuation of U.S. patent application Ser. No. 10/659,451 filed Sep. 9, 2003, now U.S. Pat. No. 7,604,821, which is a continuation-in-part of U.S. patent application Ser. No. 10/186,996 filed Jul. 1, 2002, now abandoned. Each of these patent applications are hereby incorporated by reference in their entirety.

### FIELD

Disclosed herein are methods for harvesting and processing of Porifera species, specifically sponges, and more specifically fresh water species of genus Spongilla; methods of testing and storing of the processed Spongilla powder; and methods of manufacturing and packaging of Spongilla-based topical compositions suitable for treating skin diseases.

### BACKGROUND

Skin diseases remain a significant medical and social problem throughout the world. The most common, and therefore the most significant skin diseases and conditions, include acne vulgaris, rosacea, seborrheic dermatitis, eczema (atopic dermatitis), psoriasis, photo-aging and actinic keratosis as well as wound healing and reducing the appearance of scars. These skin diseases combine to account for billions of dollars in medical treatments and untold emotional suffering. The emotional impact of skin disease is particularly relevant because patients become easy prey for unscrupulous practitioners and treatment regimes of questionable efficacy. Over the last one hundred years significant advances in pharmaceuticals and dermatological procedures have greatly reduced the severity and frequency of skin diseases. However, many patients do not comply with the often complex and tedious treatment protocols their practitioners prescribe. Moreover, the long-term use of antibiotics has resulted in increased microbial resistance of the bacteria responsible for various skin diseases. Additionally, other chemotherapies can be extremely toxic and have long term deleterious effects on the patient's health and skin. Therefore, alternative therapies for treating skin disorders that are safe, effective and easy to use are urgently needed.

Acne vulgaris is the most common of all skin disorders affecting 85% of teenagers. Nearly 80 percent of the population experience acne at some point in their lives. Moreover, in addition to being a serious medical condition, acne inflicts a heavy emotional and psychological burden on its victims. Marion Sulzberger, MD, one of the founding figures of modern dermatology, wrote in 1948 “there is no single disease which causes more psychic trauma, nor maladjustment between parents and children, more general insecurity and feelings of inferiority and general sums of psychic suffering than does acne vulgaris.” The impact can be devastating, leading to depression and even to thoughts of suicide. A survey of 1,985 people by the ASG revealed that three out of four people with acne felt depressed and almost half felt anxious. Research by William Cunliffe, MD, in the United Kingdom, showed that patients with acne had a higher unemployment rate than age- and sex-matched controls. More than a third felt they would have a better job if they didn't have acne, the survey revealed.

Acne is a chronic disease involving the pilosebaceous follicles. Sebaceous glands are found most abundantly on the face and scalp, though they are present on every part of the skin except the palms of the hands and soles of the feet. Cutaneous disorders attributed to the sebaceous gland are really disorders of the entire pilosebaceous unit. The areas most commonly involved in acne are the face, upper chest, and back. Other less common areas include the upper arms, buttocks, and upper thighs.

Acne vulgaris evolves within the pilosebaceous unit via a multifactorial pathogenesis. The central pathogenic factors in acne include excessive sebum production secondary to androgen stimulation, outlet obstruction of the sebaceous follicle arising from excess production of keratinocytes (the basic cell of the epidermis), proliferation of Propionibacterium acnes and inflammation following chemotaxis and the release of various proinflammatory mediators.

During the prepubertal period the increase in adrenal androgens triggers the enlargement of the sebaceous glands. These enlarged sebaceous glands produce increased amounts of sebum, which flows through the canal of the sebaceous follicle. This canal is lined with a keratinizing epithelium. In acne patients, there is increased production of the follicular corneocytes lining the follicle and retention of these corneocytes within the follicle. The abnormally desquamated corneocytes and the excess sebum build up within the follicle to form a microscopic, bulging mass. This enclosed, sebum-rich environment is ideal for the proliferation of P. acnes, the anaerobic bacterium that produces chemotactic factors and recruits proinflammatory molecules involved in the inflammatory phase of acne. Obstruction of the sebaceous follicle, the primary pathologic event in acne, is giving rise to the micro-comedo, the precursor of all acne lesions. It is a microscopic, bulging mass that results from a combination of hyperproliferative corneocytes and sebum and leads to follicular plugging.

Once the follicle is plugged, its lower portion becomes engorged and distended with sebaceous discharge and keratinocytes. While the pore opening remains closed, the lesion is called a closed comedo, or “whitehead.” It is a noninflammatory lesion that evolves from the microcomedo and appears as a white dot ranging from 0.1 to 3.0 mm in diameter and very slightly raised.

Oxidization occurs when the follicle enlarges enough to stretch the pore and the trapped matter is exposed to air. This causes the characteristic dark appearance of open comedones or “blackheads.” Open comedone is a noninflammatory lesion that appears as flat or slightly raised, brown-to-black color, about 3-5 mm in diameter.

Early acne, involving a majority of open and closed comedones, is a noninflammatory process. As dilation of the follicle continues, the follicular epithelium is disrupted and irritants such as sebum, hair, and keratinocytes are released into the surrounding dermis. This leakage causes an inflammatory reaction and initiates the formation of the inflammatory lesion papules, pustules, and nodules. Although P. acnes is a live bacterium, living in the follicle, it dies when the follicular structure is disrupted. Toxins are released into the dermis, which increases inflammation. Therefore, uncomplicated, inflammatory acne is a sterile process and not a skin infection. As inflammation continues to worsen, larger papules and pustules are created. A papule is a pink-to-red, raised, palpable lesion with no visible accumulation of fluid, which can range from 1 to 4 mm in diameter.

A pustule is a raised accumulation of purulent material on the skin's surface, and is similar in size to the papule. Pustules are sometimes characterized as superficial or deep. In a superficial pustule there is a localized rupture of the epithelium near the skin surface, and in a deep pustule there is extensive destruction of the entire epithelium. Acne nodules are solid, raised inflammatory lesions that exceed 6-10 mm in diameter and are situated deeper in the dermis. A nodule may persist for weeks. The acne cyst is a large nodule (may be as large as several centimeters in diameter) that has suppurated and become fluctuant. Scars form as a result of damage to the surrounding dermis. Scars may appear as small deep punched out pits (“ice pick”), atrophic macules, hypertrophic papules, or broad, sloping depressions. Darkly pigmented skin affected by acne tends to develop significant postinflammatory hyperpigmentation. This tendency has given rise to the suggestion that a new acne lesion should be designated—the acne hyperpigmented macule (AHM). The AHM can last for four months or longer, and is often the central complaint of acne patients with skin of color.

There is no single standardized grading system for acne, but there are several useful methods used to classify the disease. Most simply, acne is described as mild, moderate, or severe. Because acne is a chronic, emotionally stressful condition that may persist for years, long term therapy is often required. Presently the clinician has numerous treatment options. However, each one has significant adverse qualities and varying degrees of efficacy.

The most commonly used nonprescription topical product is benzoyl peroxide. Benzoyl peroxide (BP) is an antimicrobial that is effective for killing P. acnes. It usually takes about two weeks to work and it must be used continuously to keep acne at bay. This is because BP does not affect microcomedo formation, sebum production or the way the skin follicle cells are shed, and when patients stop using it, the acne comes back. Benzoyl peroxide is marketed under a variety of trade names in over 200 formulations, including gels, creams, lotions, washes, and bar soaps, in a variety of concentrations (most often 2.5%, 5%, and 10%). If used continuously, it often improves condition for milder cases of acne. Concentration should be chosen accordingly to skin type and tolerance. Side effects consist mainly of skin irritation including burning, blistering, crusting, itching, severe erythema, skin rash, dryness, and chemical imbalance of the skin. Benzoyl peroxide further reduces skin levels of superoxide dismutase, catalase and other skin antioxidants that are important in preventing and healing acne. Moreover, by destroying anti-oxidants naturally occurring in the skin benzoyl peroxide promotes premature aging of the skin.

Another nonprescription topical treatment is salicylic acid. Salicylic acid helps to correct the abnormal shedding of cells and is useful in treating milder acne. Salicylic acid helps unclog pores to resolve and prevent lesions. However, salicylic acid does not inhibit sebum production or possess antimicrobial properties. The patient must use salicylic acid on a regular basis to prevent acne from returning. Salicylic acid is available in many acne products, including lotions, creams, washes, gels, and pads.

In many cases over-the-counter (OTC) preparations are not effective and must be used in combination with prescription drugs. Antibiotics are the most commonly prescribed class of anti-acne medications. Antibiotics work inhibiting the growth of P. acnes and may be applied topically or taken systemically. The most widely prescribed topical antibiotics are erythromycin and clindamycin. Topical antibiotics are limited in their ability to penetrate the skin and clear more deep-seated P. acnes and do not inhibit comedo formation alone and thus must be used in combination therapies.

Systemic antibiotics circulate throughout the body and into sebaceous glands. Systemic antibiotics are used to treat severe acne but generally have more side effects than topically applied medications. They also do not address the other causative factors in acne and may take several weeks or months to clear up acne. Oral antibiotics are usually used in combination with other drugs that “unclog” follicles such as salicylic acid. However, systemic antibiotic therapy is incompatible with pregnancy and some may reduce the effectiveness of oral contraception pills, risking a pregnancy during treatment.

The front-line oral antibiotics for the treatment of acne are the tetracyclines. Tetracycline cannot be taken with food containing divalent cations such as calcium and iron and predispose patient to severe sunburn or a pruritic rash due to its photosynthesizing qualities. All tetracyclines are contraindicated in pregnancy and in children who have not yet formed their permanent teeth (risk of discoloration). Additionally tetracycline class antibiotics often cause esophageal irritation. Side effects of minocycline (a commonly prescribed synthetic tetracycline) may include vertigo, blue-gray discoloration of the skin and teeth, and a lupus-like syndrome.

Erythromycin has long been considered the preferred second-line oral antibiotic for acne therapy. It does have an excellent side-effect profile (with gastrointestinal upset generally the most common problem) and may be approved for use even in pregnant women. However, antimicrobial resistance is a major problem associated with all antibiotics commonly used to treat acne and this is most pronounced with erythromycin. The emergence of antibiotic-resistant P. acnes is an issue of increasing concern with both topical and oral antibiotics in the treatment of acne. Over the past 25 years, laboratory studies have demonstrated a rapidly increasing pattern of P acnes resistance to antibiotics, especially erythromycin (published studies indicate that the overall incidence of antibiotic-resistant P. acnes has increased from 20% in 1978 to 62% in 1996). Bacterial resistance is diminishing the effectiveness of current acne therapies and threatens to limit the options available to heal the most common skin condition diagnosed and treated by physicians. Antibiotic resistance in acne treatment is a global problem as antibiotic-resistant strains of P. acnes have been reported in the United Kingdom, Germany, France, Japan, and the United States.

Vitamin A derivatives or “retinoids” are being used with increased frequency as topical treatments for moderate to severe acne. The topical retinoids include vitamin A acid (tretinoin), its analogs, and newer agents that bind to and activate retinoid receptors. Topical retinoid preparations help to unclog pores and normalize skin growth and shedding. However, topical retinoids can cause severe skin irritation and therefore require titration at the initiation of therapy to allow patients to adjust. Moreover, topical retinoids and retinoid analogs pose a risk of teratogenicity. For example, tazarotene is a pregnancy category X drug and should not be used in pregnant women.

In 1982, the FDA approved the oral retinoid isotretinoin (ACCUTANE®). Isotretinoin is the most effective anti-acne agent on the market. It addresses three causes of acne, with beneficial effects on sebum production, keratinocytes and inflammation. However, due to the risk of severe toxicities, isotretinoin is FDA-approved only for patients who have severe, scarring, and cystic acne. Isotretinoin has numerous possible adverse reactions, including hepatotoxicity, increased levels of triglycerides (which, in turn, can trigger pancreatitis), hypercalcemia with loss of bone mass, and an increased risk of depression and suicide. Most troubling to the FDA is its most notorious risk, teratogenicity (damage to the fetus).

Anti-inflammatory medications called corticosteroids may be injected by a dermatologist directly into severe inflamed acne lesions to help heal existing lesions. However, these do not prevent development of new acne and may leave a permanent hardening in the place of injection.

Acne vulgaris is a chronic dermatologic disorder that must be treated consistently. It is not unusual that traditional topical therapy will initially worsen acne due to irritating, sensitizing, and toxic properties of the chemical therapeutic agents. This initial response usually lasts 2 to 4 weeks. Since it takes about 28 days to regenerate skin, the effect of medications does not appear immediately. Improvement, if any, becomes noticeable after 4 to 8 weeks of therapy. The maximum benefit of systemic agents, such as oral contraceptives, on acne occurs not earlier than in 3-4 months.

Many OTC preparations are toxic for the skin enzymes, which makes them automatically toxic for the skin overall. Intracellular and extracellular enzymes that found in the skin are essential for healthy skin condition, right pH and skin protective capability against pathogens. The consequences of an impairment of enzymes, even through the inactivation of trace elements that primary act for enzyme performance, cannot be evidence after one or a few applications but only following repeated treatments, for example as in the case with benzoyl peroxide preparations for acne prone skin, which might be applied several times a day for many years.

A non-compliance with anti-acne regime is one of the major reasons for treatment failure among patients with acne vulgaris. Motivating patients to adhere to treatment, especially during the maintenance phase, remains a challenge. A recent randomized, controlled study involving young adults with acne vulgaris evaluated the efficacy of various non-pharmacologic interventions for enhancing adherence to benzoyl peroxide. Adherence was measured through a combination of patient self-report and the return of self-monitoring cards. The overall adherence rate after 3 months was 48%. The study found that 52% of patients were noncompliant. They did not exactly follow the directives of their dermatologists due to the complexity of the regime.

Both researchers and practicing clinicians concur that the simpler the medication regimen for acne patients, the better the adherence. To improve the compliance among this group of the patients, effective, well tolerated, and simplified regime is needed.

Although acne is the most common skin disease and one with the greatest economical and sociological significance, it is not the only skin disorder that can benefit from improved therapeutic regimes and compositions. For example rosacea, seborrheic dermatitis, eczema (atopic dermatitis), psoriasis, photo-aging, actinic keratosis, and great number of other bacterial, viral, and fungal diseases as well as skin pigmentation disorders, wound healing, and reduction of appearance of scars are also significant health and cosmetic problems requiring improved therapies with simplified regimes.

The Holy Grail of medicine would be to slow or reverse the aging process. Aging is a complex process that is largely determined genetically. However, free radical damage caused by reactive oxygen species contributes significantly to the aging process. One manifestation of free radical-associated aging are so-called “age spots.” Age spots are actually the accumulation of special pigments called lipofuscin, a brown waste, that accumulates in the skin in highly damaged areas.

Protection against free radical-associated oxidative damage includes the activation of water-soluble reductants in the cytosol, lipid-soluble antioxidants residing in cellular membranes, and the antioxidant enzymes, superoxide dismutase, catalase, ascorbate peroxidase, glutathione peroxidase and glutathione reductase. Biogenic production of free radicals occurs mostly during normal processes of cellular metabolism. A by-product of energy metabolism is the uncoupling of electrons in the transport chain to generate superoxide, via activation of molecular oxygen, leading to the production of hydrogen peroxide and the supra-reactive hydroxyl radical. Such reactive oxygen species (ROS) are highly damaging to DNA, proteins and membrane lipids, causing cellular impairment. In the normal condition of aging, antioxidant functions decline to further accelerate the aging process, and this exacerbates the progression of age-related degenerative diseases. Therefore, preventing or decreasing the formation of reactive oxidants in metabolic electron transport presents a clear strategy for reducing cellular oxidative stress and rate of aging.

Free radical damage has also been implicated as a cause, or exacerbating factor in eczema. A recent university sponsored study examined the levels of lipid peroxidation in erythrocytes, some parameters of the antioxidant system and the activity of lysosomal enzymes in eczema patients of mix origin (exo/endogenous). The results of the study reveal an intensification of erythrocyte lipid peroxidation and a depression of antioxidant protection. This imbalance of lipid peroxidation/antioxidant systems induces modifications in biomembrane structure, especially lysosomal ones. That causes an increase of the lysosomal intracellular activity and then to a lysosomal penetration in blood circulation and facilitates cutaneous inflammatory manifestations. So, the complex treatment of eczema must include an antioxidant therapy and a pharmacological stabilization of lysosomal membranes.

The Department of Dermatology and Skin Ageing, and Cancer Research Centre of University Pavia in Italy studied the activity of 14 enzymes, representative of the main metabolic pathways in epidermis of 63 normal human subjects ranging in age from 1 month to 90 years. No difference of activity was observed in any of the enzymes studied despite the varied age. The lack of influence of age on the activity of the enzymes in human epidermis enhances the significance of the variations, which are reported in pathological conditions like psoriasis, chronic sun-damaged skin and neoplasm.

In addition, enzyme activity depression in chronically sun-exposed skin has a significantly contributes to neoplasm formation. This is clearly evidenced by the fact that the rat of cancer in skin areas usually exposed to the sun's rays (e.g., face, back of hands) are 100 times higher than on the skin of unexposed areas (e.g., abdomen). This leads one to regard chronic sun damage as a precancerous state. Chronic exposure to ultraviolet (UV) light is the leading cause of extrinsic aging, or alterations of the skin due to environmental exposure. Estimates indicate that almost half of a person's UV exposure occurs by age 18. Photo aging causes numerous histological, physiologic, biochemical and clinical changes.

One of the manifestations of aging skin is decreased ability to shed dead cells, resulting in various unsightly skin conditions. The mainstay of topical therapy of photo-aging skin continues to be chemical peels. A chemical peel is a procedure in which a topically applied wounding agent creates smooth, rejuvenated skin by way of an organized repair process. Complications of chemical resurfacing, including permanent sequelae, such as pigmentary dyschromias, infection, or scarring, may occur even though a controlled chemical wound induced.

Therefore, there remains a need for therapeutic topical compositions that are safe, effective, possess multifaceted mechanisms of action and are conducive to patient compliance.

Sponges are multicellular marine animals belonging to a large group of simple animal species known as invertebrates. Sponges originated billions of years ago and are among the oldest animals on earth. An early branching event in the history of animals separated the sponges from other metazoans. Fossil sponges dated from the late Precambrian period are among the oldest known animals and account for over 900 fossil genera. The approximately 5,000 living sponge species are classified in the phylum Porifera, which is composed of three distinct groups, the Hexactinellida (glass sponges), the Demospongia, and the Calcarea (calcareous sponges). Presently, approximately 5,000 species of sponges are known. Sponges are composed of a soft tissue suspended in a jelly-like proteinaceous matrix supported by a hard skeleton composed of needle-like structures known as spicules. Spicules are primarily composed of calcium carbonate, or silica and collagen.

Sponges belong to phylum Porifera, a highly primitive group with no tissue grade of organization. The stiff body houses numerous channels and pores allowing currents of fresh water to enter. The largest in current pores are known as oscula, and the smallest are ostia. Food-bearing water flows into the sponge through the ostia in its mound-like body and out through osculum. Water is encouraged to flow within the sponge by the action of flagellated choanocytes. Choanocytes also constitute the filter-feeding apparatus, trapping suspended food particles as they pass along the series of internal channels. Because sponges are essentially sessile, they are heavily reliant upon the effectiveness of their cell-lined channels in trapping food, oxygen uptake and removal of waste products. Sponges are highly susceptible to pollution and release of suspended sediments which block up their delicate system of tubes and pores, thus preventing basic body functions. Sponges have great powers of regeneration from injury or predation.

Two reproductive processes are known to occur in the sponges: the one of them, asexual, and the other, truly sexual. In the common fresh-water sponges, towards the autumn, the deeper layer of the sponge becomes full of exceedingly small bodies, sometimes called “seeds” or “gemmules.” The whole sponge dies down, and the seeds, enclosed in their case, remain uninjured through the winter. At the springtime, the encysted masses of sponge particles stimulated by the altered temperature of the water, creep out of their “seeds”, and grow up into a sponge.

The success rate of finding new active chemicals in marine organisms is 500 times higher than from terrestrial sources. Sponges have proven to be a prolific source of novel therapeutic agents, often with biomedical action superior to that of existing pharmaceuticals. Drug discovery from sponge colonies is now a major focus of the pharmaceutical and biotechnology industries. However, the primary area of concern about this new source of therapeutics is the question of their supply and difficulties of culturing sponges and their symbionts in the lab. The reliability and reproducibility of material from natural sources is also critical, because seasonal and environmental changes interfere with the chemical composition and biological properties of natural samples. Furthermore, biologically active molecules are expected to be produced only temporarily as a response of specific environmental stress. Extracting sufficient quantities of the active chemical from the natural source just for completion one clinical study requires many tons of sponge colonies and as a result is not a viable option for worldwide distribution. Therefore, while marine biotechnology presents tremendous potential for new pharmaceuticals, commercial success is minimal to none.

Nevertheless, like many natural products, sponges have been used in homeopathy and other forms of natural medicine for centuries. Eastern Europe and Eurasia have a long history of preparing tinctures and powders from aquatic animals including fresh water sponges. Folk medicines, known collectively as “Bardiaga,” refer to powdered fresh water sponges and used for medicinal purposes. Russian folklore and homeopathic teachings suggest that Bardiaga is useful for treating such diverse syndromes as bruising arthritis and rheumatism.

Bath sponges (Spongia officinalis) have also been used for centuries for cleaning wounds, for contraception and even as implants after breast cancer operations. Furthermore, folk medicine and natural medicine literature is replete with diverse preparations made from dried sponges and used to treat and palliate myriad diseases. The oldest medicinal sponge preparation in recorded history is dried and burned S. officinalis is offered as a treatment for goiter and thyroid-related diseases. The unusually high concentration of iodine in S. officinalis made is a uniquely effective folk remedy.

Since sponges are essentially non-motile animals that are highly susceptible to predators and changes in their microenvironments, they have evolved an elaborate bio-defense system that includes a cornucopia of biologically active (bioactive) compounds. Today, it is believed that many of these sponge-derived bioactive compounds possess cytotoxic, antibiotic, anti-viral, anti-inflammatory, and anti-fouling properties. However, most of these bioactive compounds remain uncharacterized. Furthermore, as discussed above, these bioactive compounds are produced in extremely low concentrations on a weight percent basis and therefore their isolation in pure form would require the harvesting and processing of literally tons of sponges. Many of these bioactive compounds may derive their efficacy through naturally synergistic and complementary mechanisms that would be lost if purified and studied in isolation. Therefore, the presently disclosed technology involves the development of Porifera species compositions, their formulation and applications that eliminates the need to purify and characterize individual bioactive compounds, maintains the integrity of their potentially synergistic properties and resolves the problems of supply and environmental impact.

### SUMMARY

Disclosed herein are novel skin care therapeutics derived from invertebrate species of the phylum Porifera as well as methods for harvesting the sponges and preparing the compositions.

Therefore, in one embodiment, disclosed herein is a method of preparing a Spongilla powder for the preparation of a therapeutic composition for the treatment of skin conditions, the method comprising (a) sampling fresh-water sponges of the species Spongilla lacustris from a body of fresh water in the Astrakhan region of Russia during a potential harvest period, (b) determining a bioactivity profile of the S. lacustris sampled in step (a) to establish a harvest period, (c) recording the environmental conditions at the harvest location during the time period established in step (b), wherein the environmental conditions are air temperature, water temperature, relative humidity, precipitation, wind speed, salinity, and oxygenation of the body of fresh water at the harvest location, (d) harvesting the S. lacustris during the harvest period established in step (b), (e) removing gross contamination from the harvested S. lacustris, (f) washing the harvested S. lacustris, (g) drying the harvested S. lacustris for a period of time not to exceed 21 days from the day of harvest, (h) grinding and sieving of the dried S. lacustris to form a particle size less than 2 mm and repeating the grinding and sieving process to form a substantially pure Spongilla powder having an average particle size of less than 0.2 mm, and (i) analyzing the resulting substantially pure Spongilla powder to provide a Certificate of Analysis for each lot of Spongilla powder describing the lot's composition, purity and activity to yield reproducible lots of substantially pure Spongilla powder suitable for clinical use. In another embodiment, determining the bioactivity profile comprises performing high-performance liquid chromatography and at least one assay of a sebocyte proliferation assay, a keratinocyte proliferation assay, and an anti-inflammation assay on the sample, and wherein the harvest period is established if the bioactivity profile of the sample is substantially equivalent to a previously established preferred bioactivity profile.

Also disclosed herein is a method of preparing a Spongilla powder for the preparation of a therapeutic composition for the treatment of skin conditions, the method comprising harvesting fresh-water sponges of the species Spongilla lacustris from a body of fresh water in the Astrakhan region of Russia during a harvest period, removing gross contamination from the harvested S. lacustris, washing the harvested S. lacustris, drying the harvested S. lacustris for a period of time not to exceed 21 days from the day of harvest, grinding and sieving of the dried S. lacustris to form a particle size less than 2 mm and repeating the grinding and sieving process to form a substantially pure Spongilla powder having an average particle size of less than 0.2 mm, and analyzing the resulting substantially pure Spongilla powder to provide a Certificate of Analysis for each lot of Spongilla powder describing the lot's composition, purity and activity to yield reproducible lots of substantially pure Spongilla powder suitable for clinical use.

In another embodiment, the method further comprises storing the dried S. lacustris material before grinding. In yet another embodiment, the harvest period is the summer of each year or the fall of each year. In another embodiment, the harvest is halted if the environmental conditions change more than about 20% during the harvest period.

In yet another embodiment, the harvested S. lacustris is dried outdoors, such as near the harvest location. In another embodiment, the harvested S. lacustris is dried at an ambient temperature of more than about 60° F. In another embodiment, the harvested S. lacustris is dried at an ambient relative humidity of below about 90%. In other embodiments, the harvested S. lacustris is dried for at least about 14 days, at least about 18 days, or about 21 days.

In another embodiment, the dried S. lacustris has a residual moisture content of about 0.1% to about 10%, from about 0.1% to about 5%, or from about 0.1% to about 2%.

In another embodiment, the sieving step comprises passing the ground S. lacustris through a series of sieves with progressively smaller apertures wherein the last aperture is about 0.2 mm.

In another embodiment, the skin condition is acne vulgaris, rosacea, seborrheic dermatitis, atopic dermatitis, psoriasis, photo-aging, or actinic keratosis. In another embodiment, the skin condition is wound healing or reducing the appearance of scars.

Also disclosed herein is a therapeutic composition for treating skin conditions consisting of a substantially pure powder of a fresh water the substantially pure powder of S. lacustris and at least one pharmaceutically acceptable excipient, wherein the substantially pure powder of S. lacustris has a maximum particle size of about 0.2 mm. In another embodiment, the skin condition is acne vulgaris, rosacea, seborrheic dermatitis, atopic dermatitis, psoriasis, photo-aging, wounds, scars, or actinic keratosis. In yet another embodiment, the skin condition is wound healing or reducing the appearance of scars.

In another embodiment, the S. lacustris is harvested from the Astrakhan region of Russia. In another embodiment, the pharmaceutically acceptable excipient is water, glycerin, gels, oils, waxes, emollients, cleansers, fragrances, antiseptics, anesthetics, seaweed powder, coral powder, hydrogen peroxide, enzyme gel, jojoba oil, or boric acid. The water can be water for injection, irrigation water, distilled water, deionized water, or floral water. In another embodiment, the substantially pure powder of S. lacustris comprises approximately 50% to 60% insoluble material.

Also disclosed herein is a method of treating skin conditions comprising applying to the skin of an individual a therapeutic composition consisting of a substantially pure powder of Spongilla lacustris, and at least one pharmaceutically acceptable excipient, wherein the substantially pure powder has a maximum particle size of about 0.2 mm, and wherein the therapeutic composition treats the skin condition. In another embodiment, the skin condition is acne vulgaris, rosacea, seborrheic dermatitis, atopic dermatitis, psoriasis, photo-aging, wounds, scars, or actinic keratosis. In yet another embodiment, the skin condition is wound healing or reducing the appearance of scars.

In another embodiment, the S. lacustris is harvested from the Astrakhan region of Russia. In another embodiment, the pharmaceutically acceptable excipient is water, glycerin, gels, oils, waxes, emollients, cleansers, fragrances, antiseptics, anesthetics, seaweed powder, coral powder, hydrogen peroxide, enzyme gel, jojoba oil, or boric acid. The water can be water for injection, irrigation water, distilled water, deionized water, or floral water. In another embodiment, the substantially pure powder of S. lacustris comprises approximately 50% to 60% insoluble material.

In another embodiment, the therapeutic composition consists of substantially pure powder of S. lacustris and 3% hydrogen peroxide.

In yet another embodiment, the substantially pure powder of S. lacustris comprises approximately 50% to 60% insoluble material. In another embodiment, the composition abrades the skin of the individual. In another embodiment, the method results in resurfacing of the skin of the individual. In another embodiment, the therapeutic composition is massaged into the skin such that spicules contained in the substantially pure powder of S. lacustris penetrate the upper layer of epidermis. In another embodiment, the method results in debriding of the skin of the individual. In another embodiment, the method results in increased cell turnover of the skin of the individual.

Also disclosed herein is a method of inhibiting chemotaxis comprising applying to a tissue in need of reduction of chemotaxis a therapeutic composition consisting of a substantially pure powder of Spongilla lacustris, and at least one pharmaceutically acceptable excipient, wherein the substantially pure powder has a maximum particle size of 0.2 mm, and wherein the therapeutic composition reduces chemotaxis.

Also disclosed herein is a method of reducing inflammation comprising applying to a tissue in need of reduction of inflammation a therapeutic composition consisting of a substantially pure powder of Spongilla lacustris, and at least one pharmaceutically acceptable excipient, wherein the substantially pure powder has a maximum particle size of 0.2 mm, and wherein the therapeutic composition reduces the inflammation. In another embodiment, the S. lacustris is harvested from the Astrakhan region of Russia. In another embodiment, the pharmaceutically acceptable excipient is water, glycerin, gels, oils, waxes, emollients, cleansers, fragrances, antiseptics, anesthetics, seaweed powder, coral powder, hydrogen peroxide, enzyme gel, jojoba oil, or boric acid. The water can be water for injection, irrigation water, distilled water, deionized water, or floral water. In another embodiment, the substantially pure powder of S. lacustris comprises approximately 50% to 60% insoluble material.

### BRIEF DESCRIPTION OF THE FIGURES

FIG. 1 depicts a specific geographical location where Spongilla lacustris can be harvested.

FIG. 2A-D depicts patients before (FIGS. 2A and 2B) and after (FIGS. 2C and 2D) receiving treatment for acne with the disclosed Spongilla composition.

FIG. 3A-B depicts a patient before receiving treatment for wound and scar treatment (FIG. 3A) and seven days following a single treatment (FIG. 3B) with the disclosed Spongilla composition.

FIG. 4A-B depicts a patient before receiving treatment for actinic keratosis (FIG. 4A) and seven days following a single treatment (FIG. 4B) with the disclosed Spongilla composition.

FIG. 5A-B depicts a patient before (FIG. 5A) and after (FIG. 5B) receiving an anti-inflammatory treatment with the disclosed Spongilla composition.

FIG. 6A-B depicts a patient before (FIG. 6A) and after (FIG. 6B) receiving a post-inflammatory hyperpigmentation treatment with the disclosed Spongilla composition.

FIG. 7A-B depicts a patient before (FIG. 7A) and after (FIG. 7B) receiving resurfacing treatment with the disclosed Spongilla composition.

FIGS. 8A and 8B depicts harvesting of S. lacustris.

FIG. 9 depicts drying of S. lacustris.

FIG. 10 depicts a scanning electron micrograph of S. lacustris (Volga River specimen) ectosomal skeleton (60×).

FIG. 11A-B depicts a scanning electron micrograph of S. lacustris (Volga River specimen) choanosomal skeleton at 130× (FIG. 11A) and 500× (FIG. 11B) magnification.

FIG. 12A-B depicts a scanning electron micrograph of S. lacustris (Volga River specimen) megascleres (spicules) at 100× (FIG. 12A) and 8000× (FIG. 12B) magnification.

FIG. 13 depicts a scanning electron micrograph of S. lacustris (Volga River specimen) microcleres (spicules) at 3000× magnification.

FIG. 14 depicts the inhibition of human primary sebocyte proliferation by the disclosed S. lacustris compositions in a dose-dependent manner.

FIG. 15 depicts the effects of different lots of the disclosed S. lacustris compositions compositions on keratinocyte proliferation.

FIG. 16 depicts bacterial growth in the presence of Spongilla extracts.

FIG. 17A-B depicts the activity of extracts of Spongilla powder extracted at room temperature in water, ethanol, and hydrogen peroxide and tested at 1:40, 1:80, and 1:150 dilutions (FIG. 17A) or 1:50, 1:150, and 1:450 dilutions (FIG. 17B).

FIG. 18A-B depicts the activity of extracts of Spongilla powder extracted at 60° C. in water, ethanol, and hydrogen peroxide and tested at 1:40, 1:80, and 1:150 dilutions (FIG. 18A) or 1:50, 1:150, and 1:450 dilutions (FIG. 18B).

FIG. 19A-B depicts the activity of concentrated (1×, 2×, 4×, 10× and 30×) extracts of Spongilla powder extracted at room temperature (FIG. 19A) and at 60° C. (FIG. 19B) in water, ethanol, and hydrogen peroxide.

FIG. 20 depicts the effects of Spongilla extracts on chemotaxis of THP-1 cells. MCP-1 (10 ng/ml) was placed in the lower wells of chemotaxis chambers. In the upper wells, THP-1 cells (5×10⁵) were placed. 13-Cis RA or a 1:50 or 1:100 dilutions of 15× extract were placed in both compartments. The chambers were incubated for 3 hr. Migration Index was calculated as the number of cells migrated in the presence of the ligand divided by the number of cells migrating in the presence of buffer only (control CON). P values are placed on top. Mean±SEM of 5 wells each.

FIG. 21 depicts the anti-inflammatory effects of Spongilla on interleukin-8-induced neutrophil migration.

FIG. 22A-B depicts the HPLC analysis of Lot #1 at 210 nm (FIG. 22A-1-22A-4) and 254 nm (FIG. 22B-1-22B4).

### DETAILED DESCRIPTION

Natural chemistry of various Porifera species is a critical part of the methods of harvesting of Spongilla species for isolation, identification and preservation of the vast array of novel molecules and compounds produced by Spongilla at various stages of life cycle. These molecules and compounds are biologically active metabolites that are produced by Spongilla for a range of purposes including relief from environmental stress, chemical signaling, and aggression among species. Many of these molecules and compounds possess structural features that are unique to the sponge species from a specific geographic region and result from the unique aquatic environment (high level of halogens and nitrogen) in which the sponge species live. The bioactivity of these molecules and compounds strictly depends on the time of harvesting, methods of processing and preservation.

Fresh water sponges including, but not limited to, Spongilla lacustris L., Spongilla fragilis Leidy, and Ephydatia fluviatilis, are not presently used to provide pharmaceutical compositions that have undergone rigorous safety and efficacy testing. Poriferans are natural sources of complex biologicals that provide new compositions useful for treating, palliating and preventing a variety of diseases, including, but not limited to, acne vulgaris, rosacea, seborrheic dermatitis, eczema (atopic dermatitis), psoriasis, photo-aging and actinic keratosis as well as wound healing and reducing the appearance of scars. The synergetic poly-pharmacy of these complex biologicals has advantages over synthetic single-ingredient drugs by providing greater therapeutic benefit and less overall toxicity. Specifically, and not intended as a limitation, but merely as an exemplary embodiment of the invention, the present inventor has developed therapeutic compositions derived form the fresh water sponge species Spongilla lacustris L.

Disclosed herein is an anti-acne vulgaris therapeutic is provided that effectively treats, palliates, and in some cases prevents the most important pathological factors in acne development.

Acne comes about from a straightforward and well-understand series of steps. There are a limited and defined number of points where treatments can intervene. Even so, there are dozens of treatments available, both prescription and non-prescription. Treatments are targeted at interrupting the four steps in the process: a) excess sebum production; b) the rapid proliferation of Propionibacterium acnes; c) the rapid shedding of keratinocytes; and d) the release of inflammatory mediators and the resultant immune response. (Table 1).

TABLE 1

| Point of Intervention | Therapeutic Agent |
|---|---|
| Excess sebum production | Oral contraceptives, isotretinoin |
| Proliferation of P. acnes | Topical and oral antibiotics, |
| | topical benzoyl peroxide |
| Shedding of keratinocytes | OTC: Topical benzoyl peroxide, |
| | topical salicylic acid |
| | Prescription: Topical benzoyl |
| | peroxide, topical retinoids, |
| | isotretinoin |
| Release of inflammatory | Corticosteroid injections into the |
| mediators | lesion, isotretinoin, oral antibiotics |

The anti-acne vulgaris therapeutics disclosed herein act at all of these points of intervention. They inhibit proliferation of human sebocytes, thereby reducing sebum production; inhibit growth of P. acnes; inhibit proliferation of keratinocytes, and, increase shedding of keratinocytes, which leads to unclogging of pores. Clinical evidence suggests that this therapeutic also produces a direct and indirect anti-inflammatory effect. Meanwhile, pre-clinical studies and human use have shown it is completely non-irritating and non-sensitizing.

The Spongilla-derived therapeutics disclosed herein, if derived from Spongilla lacustris harvested under certain conditions at summer and processed and stored in accordance with the disclosure herein, consists of multiple active compounds with multiple biological activities, and counter all of the mechanisms by which acne occurs, which accounts for its impressive efficacy as a treatment for acne.

Moreover, the ingredients of the anti-acne preparations disclosed herein can act synergistically via additional one or more of following mechanisms. The compositions stimulate a localized histamine reaction that dilates blood vessels thus increasing blood flow to the treatment area. This results in increased amounts of oxygen, nutrients, and antibodies reaching the skin cells. Due to the stimulation of blood circulation and lymphatic drainage, the removal of excess fluid, bacteria, and debris is increased. Continuous use the topical therapeutics reduces skin fatty acid concentrations and normalize keratin turnover in the sebaceous follicles.

Furthermore, refined organic residue, such as, but not limited to skeletal spicules, mechanically separate epidermis surface layers and reduces the keratinocytes cohesion thereby increasing stratum corneum sloughing and sebum plug and loose keratinocyte removal, which opens pores and prevents future occlusion and consequent formation of comedones.

Multiple acne infections over time change the chemical balance of the skin and ultimately changes the balance of the body's chemistry. For example, people with moderate to severe acne have significantly less zinc in their body than people their age that do not have acne. Furthermore, chronic acne sufferers have skin that is uniquely deficient in linoleic acid and protective antioxidants.

The disclosed compositions correct these imbalances allowing the skin's immune processes to effectively control bacteria and prevent infections. Accordingly, in one embodiment compositions are prepared having high bioactivity and contain high concentrations of zinc, linoleic acid, antioxidants, calcium and other biochemicals that act to block the conditions that lead to acne and facilitate the healing.

When used as a peeling agent, the Spongilla composition rejuvenates the skin, stimulates new cell growth, elastin and collagen production and improves skin tone and texture. The enzymes contained in the compositions dissolve and digest old, debilitated or dead cells from the skin's outer layer without harming the younger, living cells and result in softer, smoother skin. Overall, the compositions help to dissolve stagnant spots, infiltrates, remove superficial scars comedones, regulate skin pH and sebum production and prevent further acne eruption and scar formation. Furthermore, significant clinical evidence supports the conclusion that the Spongilla therapeutic preparations disclosed herein also increase cell turnover and prevent skin cancer.

The compositions disclosed herein are effective in treating dermatologic conditions including, but not limited to, mild, moderate, and severe acne, rosacea, seborrhea, seborrheic dermatitis, eczema (atopic dermatitis), photo-aging, actinic keratosis, wound healing and reduction of the appearance of scars, prevention and treatment of post-traumatic bruising and swelling, enlarged pores, inflammation, post-traumatic and post-inflammatory hyperpigmentation, uneven skin surface, dry skin, wrinkles, sun damage, burns, cuts and scrapes, folliculatis, hair loss, herpes simplex infection, hives, ingrown hair, keratosis pilaris, melasma, psoriasis, psoriatic arthritis, rhinoplasty, facelift, shingles, prevention and treatment of basal cell carcinoma, and prevention of other skin cancers.

Furthermore, the compositions disclosed herein can be used safely with other therapies for acne and other dermatological diseases including but not limited to traditional antimicrobial scrubs, astringents, salicylic acid preparations and other topical and systemic therapies including but not limited to antibiotics, retinoids and anti-inflammatory over-the-counter and prescription drugs.

Over-the-counter preparations and prescription pharmaceutical preparations of Spongilla compositions are both considered within the scope of the present invention. Moreover, the compositions are undergoing clinical trials and are intended for use in a professional environment administered and used under the direction of a qualified physician. As such the compositions may also include instructions for use and product labeling approved by the United States Food and Drug Administration (USFDA) and other healthcare regulatory agencies world-wide. In one embodiment product labeling and instructions for use that comply with all applicable sections of 21 U.S.C. Chapter 9, Subchapter V, part A section 352 and section 21 CFR part 201 (hereinafter referred to as FDA approved product labeling and/or package insert) are provided.

The Spongilla compositions have been analyzed extensively. The desiccated and granulated raw material of the present invention is an odorless, grayish-red non-hygroscopic powder. The powder is partially soluble in water and forms a greenish-red colored solution when mixed in a ratio of 1 part to 3; approximately 50 to 60% percent remains insoluble and comprises the organic fraction providing compositions of the present invention with mechanical-abrasive properties. The pH of the soluble fraction is between approximately 7.0 to 7.5 with a mean pH of 7.35; the specific gravity is between approximately 1.04 to 1.07 with a mean specific gravity of 1.058. Peak absorption is observed at between 210 nm to 250 nm when measured between 200 and 900 nm using methods known to those skilled in the art of physical chemistry.

Table 2 includes a non-limiting representative analysis of the organic and inorganic constituents.

TABLE 2

| Inorganic | IC mg/g | Organic | OC g/110 g | Enzyme | Units |
|---|---|---|---|---|---|
| Component | of dried | Component | of dried | Activity | per |
| (IC) | raw material | (OC) | raw material | (EA) | 100 g |
| Sodium | 160-170 | Protein | 1.90-2.00 | Alkaline | 80-90 |
| | | | | Phosphatase | |
| Potassium | 120-130 | Neutral | 1.10-1.20 | Asparagine | 20-25 |
| | | fats | | Transferase | |
| Ammonia | 30-40 | Glucose | 0.3-0.4 | Alanine | 9-10 |
| | | | | Transaminase | |
| Calcium | 160-170 | Steroids | 0.0002 | Gamma-glutamyl | 7-8 |
| | | | | Transpeptidase | |
| Magnesium | 20-40 | Hydroxy- | Trace | Catalase | 50-55 |
| | | purines | | | |
| Iron | 320-330 | Total | 0.012-0.014 | Malanic | 0.15-0.2 |
| | | Nitrogen | | dialdehyde | |
| Copper | 190-200 | | | Superoxide | 6030-6040 |
| | | | | dismutase | |
| Zinc | 11-13 | | | Ceruloplasmin | 450-500 |
| Chlorine | 130-140 | | | | |
| Sulfate | 115-120 | | | | |
| Phosphate | 420-430 | | | | |
| Nitrate | 25-30 | | | | |
| Bicarbonate | 540-550 | | | | |
| Carbonate | 120-125 | | | | |
| Silicates | 13-15 | | | | |

* Ranges are approximate and based on normal laboratory standard deviations for the assay methods employed. Assays were conducted using standard analytical procedures known to those having ordinary skill in the art of analytical chemistry. Enzyme activity is expressed in units of enzyme activity per 100 mg of dried material. Units are based on a recognized International Biological Standard for each assay.

It should be understood by those skilled in the art that the elemental and organic analysis performed on representative samples is not intended to be a comprehensive or even partial listing of the active ingredients found in the compositions. As previously discussed, there may be myriad bioactive molecules present in the Porifera products that have not been previously identified. The analytical data in Table 2 provides persons skilled in the art non-limiting data that may be useful in characterizing compositions. However, in addition to other possible synergetic and complementary bioactive compounds contained in the compositions, the ingredients identified in Table 2 may also provide certain beneficial effects. Without being bound to this theory, the present inventor proposes a possible role for many of the quantified ingredients in Table 2.

The Medical Research Council in Dunn Clinical Nutrition Centre, Cambridge, United Kingdom conducted the study showing dissimilar nitrate reduction by P. acnes isolated from human faces. Low concentrations of nitrite (ca. 0.2 mM) inhibited growth of P. acnes in culture. The nitrite was slowly reduced to nitrous oxide enabling growth to occur, suggesting that denitrification functions as a detoxification mechanism.

Copper is involved in the production of collagen, the protein responsible for the structural integrity of bone, cartilage, skin, and tendon. It is also involved in the production of elastin, the protein that is mainly responsible for the elastic properties of blood vessels and skin. Studies have proven that copper is also essential to tissue building processes. As we age, our skin thins, and lines and wrinkles develop as our bodies become slower to produce collagen, elastin, and glycosaminoglycan (GAG). GAG functions as cement that bonds tissue components together. Age spots appear and skin becomes dull and lifeless as cell renewal slows and the skin retains less moisture. Scientific studies have demonstrated that copper plays a vital role in skin health, by helping restore the skin's ability to repair itself. Copper is a powerful collagen and elastin promoter and plays an antioxidative role in the body. It is important in the production of GAG. Copper-dependent enzymes increase the benefits of natural tissue building processes.

Zinc, through a group of enzymes called metalloproteinases, breaks down dysfunctional tissues of acne, thereby enabling the infection site to rebuild. Zinc directs the body's T-cells to bacteria and infection by way of a signaling chemical called adenosine deaminase. Zinc is a key element in the production of new skin cells, new collagen and elastin, new blood vessels and other components of the skin. Moderate to severe acne literally consumes the body's supply of zinc, causing the patient to become systemically zinc deficient. When the skin is zinc deficient, the clean up and repair of infections can be slow and possibly incomplete, allowing the potential of scarring. Zinc is especially critical with cystic acne because cystic infections do not discharge waste materials.

Zinc in sufficient amounts and right form acts to prevent acne as well. Testosterone in the skin converts to dihydrotestosterone, which stimulates the production of sebum and contributes to acne. Zinc via 5-alpha-reductase inhibition blocks this conversion and thereby reduces sebum production. Zinc is required in the production of the skin's super antioxidants that reduce the damage of free radicals, reducing inflammation and keeping the healing process moving forward.

Glucocorticoids are widely used for the treatment of various diseases, despite known side effects such as skin atrophy. Many studies have shown that the status of collagen fibers in the skin is affected by glucocorticoid treatment. The results of a study in Japan showed that skin treatment with glucocorticoids strongly interferes with both the synthesis and degradation of type I collagen and, more drastically, type III collagen, the molecule that is known to play a major role in the initiation of wound healing. The study provided a molecular basis for the deterioration of skin function, impaired wound healing, and skin atrophy caused by glucocorticoid treatment. Contrary to experience with synthetic steroids, naturally occurring steroids contained in the present invention provide excellent anti-inflammatory effects without adverse properties described above.

Enzymes are specific biological catalysts in the skin. Failure in the production or activity impairment of a single enzyme leads to metabolic disorders and worsen the acne condition. Since activity of many enzymes is significantly depressed in skin with metabolic diseases, photo-aging and cancer, their presence in therapeutics is the most desirable for the treatment of the above mentioned diseases.

Alkaline phosphatase (AAP) is a single enzyme of the ‘bone-liver-kidney’ type, which is present both in a soluble and in membrane-bound form in the skin. It occurs almost exclusively in the dermis, not more than 1% of the total alkaline phosphatase of human skin being present in the epidermis.

In a study that was carried out on leukocyte enzyme activity from prints of skin cut wounds, cyto-chemical analysis revealed a rapid increase in enzyme activity in the fourth hour after the wound occurred, which can be explained by the alteration in leukocyte metabolism induced by the damaging agent. Thus suggesting a critical role for AAP in wound healing.

Asparaginase is enzyme that has proved to be particularly promising for the treatment of cancer. Its action depends upon the fact that tumor cells are deficient in aspartate-ammonia ligase activity, which restricts their ability to synthesize the normally non-essential amino acid L-asparagine. Therefore, they are forced to extract it from body fluids. The action of the asparaginase does not affect the functioning of normal cells which are able to synthesize enough for their own requirements, but reduce the free exogenous concentration and so induces a state of fatal starvation in the susceptible tumor cells. A sixty percent incidence of complete remission has been reported in a study of almost 6,000 cases of acute lymphocytic leukemia.

Gamma-glutamyl transpeptidase (GGT) is another enzyme critical in antioxidant and anticancer defense. GGT activity was found in both the epidermis and dermis, the former being more active. GGT is one of the most studied chemicals in cancer chemoprevention, a desirable and important facet of biomedical research.

Superoxide dismutase (SOD) is the best known and perhaps most important of the antioxidant enzymes. It converts the very harmful free radicals super oxide to the less active peroxide, which is then further converted by another antioxidant enzyme, catalase (CAT), into water. The natural synergetic interaction between these two antioxidant enzymes constitutes the most effective system of free radical control in our bodies. Their combined activity represents a major anti-aging factor. Deficiency in SOD/CAT is the most notorious factor in most inflammatory processes.

Research suggests that SOD may be the most important enzyme involved in free radicals scavenging and marinating cell membrane integrity. Compositions containing SOD/CAT have demonstrated utility as pre- and post operative supplements. When administered to surgical patients significant improvement in recovery rates and reduced convalescent periods have been observed. Moreover, when used as a therapeutic SOD can exert strong regenerative effects on tissues that have become hardened or fibroid because of age, disease, or injury.

In a study conducted by the Department of Dermatology, Suleyman Demirel University Faculty of Medicine, Isparta in Turkey, researchers investigated the role of reactive oxygen species in inflammation of acne by determining the activity of antioxidant defense enzymes in leukocytes. The results showed that activity of SOD was significantly decreased in the acne group. Researchers suggested drugs with antioxidative effects are valuable in treatment of acne patient's, since their antioxidative defense enzymes are severely impaired.

Ceruloplasmin (CP) is a copper-containing protein that is an important extra-cellular antioxidant and free radical scavenger. The liver is the primary organ that expresses CP; however, recent studies have identified the lung as another major site of CP synthesis. Ceruloplasmin plays critical role in host defense against oxidative damage and infection.

In an exemplary embodiment the Porifera is used to prepare topical therapeutics is Spongilla lacustris. As discussed briefly above, crude preparations of fresh water sponge colonies of mixed genus, including but not limited to Spongilla lacustris L., Spongilla. fragilis Leidy, and Ephydatia fluviatilis, have been used by native people to prepare folk remedies (e.g. Bardiaga) for centuries. However, these crude preparations generally comprise inconsistent mixture of various sponge genus, myriad contaminates including other marine life forms, soil sediment and other debris associated with the sponges' natural habitat. It is produced without batch-to-batch consistency, necessary for pharmaceuticals. Moreover, sponge colonies were collected randomly without regard to environmental conditions such as, but not limited to, the presence or absence of predators, water temperature and water pressure, oxygen availability, salinity, season and life cycle. Consequently, these compositions, like many other crude natural products were seldom efficacious and often dangerous to use. Unlike crude folk remedies the therapeutic compositions of the present invention comprise substantially pure Spongilla powder. As used herein, “substantially pure” refers to a natural product, specifically a Porifera sp. that has been separated from environmental debris including rocks, sticks, other marine life etc., washed, dried, ground, sieved and sized.

For botanical products to be manufactured as medicinal compounds, the World Health Organization (“WHO”) has published GACP as guidelines for handling raw materials during harvesting and initial processing. The present inventor has determined through analysis and clinical research that harvesting conditions, collection practices for Spongilla harvesting, testing for quality control (safety and efficacy), and formulation protocols are important in providing a reproducibly safe and effective topical therapeutic products.

Disclosed herein are methods for 1) harvesting and processing of Porifera species, specifically sponges, and more specifically fresh water species of genus Spongilla; 2) testing and storing of the processed Spongilla powder; and 3) manufacturing and packaging of Spongilla-based topical compositions that are suitable for treating skin diseases. Primary elements include preventing material from being in contact with soil during drying, use of equipment and processing facilities that are clean and free of contamination, use of unique numbers to identify and track production batches, and documentation of all material processing, testing, and manufacturing activities and parameters.

Fresh water sponges are easily identified by competently trained marine biologists possessing no more than ordinary skill. For example, when a fresh-water aquatic environment is observed Spongilla appears as dull creamy brown to medium brown amorphous bodies. Often times larger sponge colonies will appear greenish due to algae trapped within the sponges' bodies. Furthermore, evidence of sponge viability and bioactive compound excretion can be observed empirically and include such factors as the lack of algal overgrowth and low predation rate.

Spongilla lacustris is generally preferred for making the disclosed compositions because this sponge species is highly tolerant of natural environmental variation and grows extremely well in a wide range of habitats. In order to avoid collecting environmentally induced variants having less than ideal potency and safety, S. lacustris is preferably collected at summer's end (during or close to the last two weeks of August) on warm sunny days (when colonies of adult sponges are giving birth to live larvae) or at fall (September-October, in non-reproductive “resting” stage). The season of choice is determined by target bioactivity of metabolites produced by sponges at different seasons.

For example, the sponge activity preferred for a specific condition such as acne is known by comparison of bioassay results (sebocyte and keratinocyte proliferation assays, anti-inflammatory assays, and the like) with high-performance liquid chromatography (HPLC) profiles of the sponge, a process referred to as “fingerprinting”. Once the HPLC fingerprint for a condition is known, sponges are sampled periodically to determine the target season. Once the target season is determined, the sponges are sampled within the target season until the sponge population exhibits the desired “fingerprint”. At that time the harvesting begins. The sponges are continually analyzed during the harvest period and if the “fingerprint” changes by 20% or more, the harvest is halted and only those sponges harvested having the preferred characteristics are used. In other embodiments, the harvest is halted if the fingerprint changes by 15% or more, or 10% or more. In one embodiment, the target season is the summer. In another embodiment, the target season is the fall.

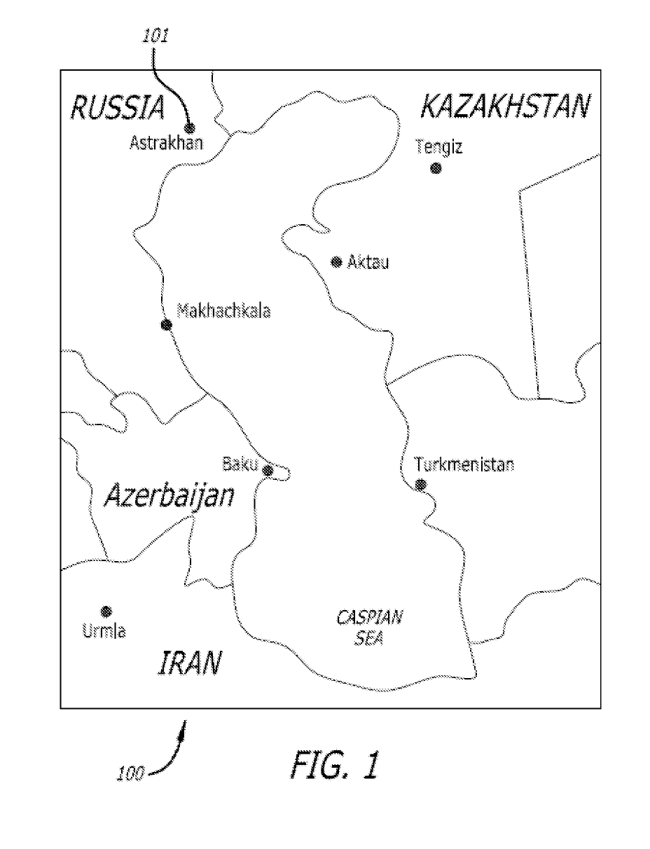

Aquatic environments favorable to S. lacustris production include an identifiable substratum having submerged rocks, sticks and branches. Generally, lakes are better natural habitats than rivers and streams for the development of large sponge colonies due to the absence of strong currents. In still waters such as lakes, freshwater sponges form colonies ranging from 2.4 to 40 cm across in deer-horn-shape, finger- and bush-like forms. Water clarity is also an important environmental factor in supporting large, developed Spongilla lacustris colonies. Water clouded by dirt, mud and dissolved solids depresses sponge growth thus reducing colony size and sponge quality. Consequently, muddy, cloudy and turbulent waters, as well as lakes having contaminated source waters should be avoided when selecting harvest locations. In one embodiment of the present invention S. lacustris is harvested from fresh water lakes in the Russian Federation northwest of the Caspian Sea, specifically Astrakhan region as depicted in FIG. 1.

Once an appropriate aquatic environment and sponge habitat (the territory) is identified, sponge collection can begin using methods commonly known to those skilled in the art of marine biology. For example, sponges can be collected manually using basic under water diving techniques, or in deeper waters larger colonies are harvested using the Agassiz trawl (AGT) or epibenthic sledge (EBS). However, sponges smaller than 0.5 cm in diameter are unlikely to be collected by AGT.

Freshwater sponges usually appear branched or clumped. Under certain environmental conditions S. lacustris colonies occur in a thin crust-like carpet several meters across and must be collected manually, with fork-like tools, and nets.

If S. lacustris is harvested for the manufacturing of skin therapeutics on an annual basis, the annual lot-to-lot consistency is critical for safety and efficacy. Only harvesters duly trained and licensed to harvest Spongilla in the territory are allowed to participate in a harvest. Prior to the commencement of each annual harvest, harvester(s) receive comprehensive instructions concerning the specifications as contained in a harvesting protocol, understand such specifications and acknowledge that failure to comply with specifications will result in rejection of harvested Spongilla.

To minimize the day-to-day chemical variation of biometabolites produced by sponges, it is preferred that harvest period is about seven consecutive days (a single week) and does not exceed a period of fourteen consecutive days. Environmental conditions such as outside air temperature, water temperature, relative humidity, precipitation and wind speed are closely monitored and recorded every 12 hours starting July 1, during each annual harvest and desiccation period, and one month after completion of drying of each annual harvest. Environmental conditions such as oxygen availability and salinity are also recorded. Samples of the sponges' natural chemistry are periodically tested for the bioactivity of interest. Once the bioactivity of interest is detected in desirable concentration, the harvest begins. If, during the harvest, sudden environmental changes occur, harvesting is terminated, because the environmental changes can lead to changes in the natural chemistry of the sponge.

During the harvest, the harvesting personnel strictly adhere to a formal and documented system to assign batch or lot numbers in accordance with the following specifications.
A unique lot number and a written record for each delivery by each harvester is assigned, to identify a specific lot with the individual who harvested such lot. In addition to the unique number, the documentation includes identification by name of the harvester; date(s) and location of harvest; dates and location of drying; weight; the results of the initial screening; and other information as may be necessary.
A unique lot number is assigned to groups of harvester lots of Spongilla, which are initially screened to allow traceability back to the harvesters' individual lots of such material of Spongilla.
A unique lot number is assigned to each lot of Spongilla milled into a powder which permits traceability to the lots of Spongilla initially screened and to the harvesters' of such lots. This number also is the ‘final’ lot or batch number.

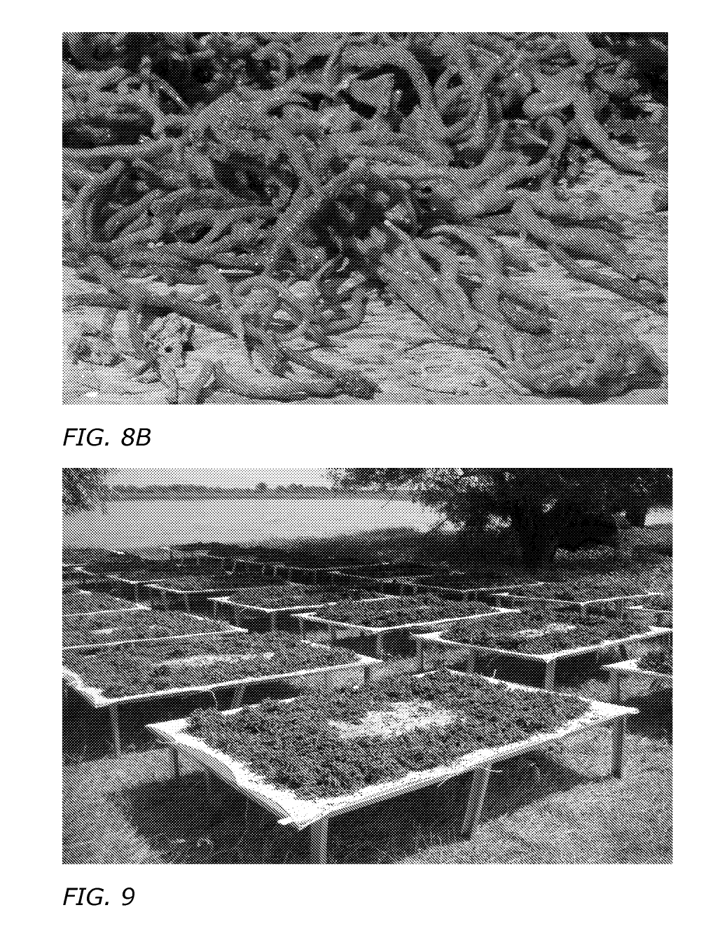

Freshly collected S. lacustris removed from their aquatic habitat of the Astrakhan region appear as brunched masses and emit a characteristic odor that most observers describe as unpleasant (FIGS. 8A and 8B). Before the collected sponge mass is dried it must be clean of gross contamination including portions of the substrata, shells, stems, plants, small fresh water animals, rocks and other impurities. Next the sponge mass is washed to remove dirt, sand, silt and soluble impurities. The wash water is changed repeatedly until it is clear and the sponges appear free from contamination. After removing gross debris and cleaning, the sponge mass is weighed and dried. Drying is preferable done in the open air on a warm clear day near the harvesting location.

Spongilla desiccation is performed in common drying areas that are used exclusively for Spongilla desiccation and are fenced to preclude animal entry or unauthorized human entry. In no event is the drying area located where household animals have access. Necessary security is provided over common drying areas during the sponge desiccation period to prohibit poaching or animal contact. The common drying areas is protected from wind through the use of windbreaks as necessary to limit the exposure of the Spongilla to contaminants.

Additional common drying areas of appropriate size are created so that such areas exist near to each and all harvesting locations.

The common drying areas are large enough to accommodate desiccation of the entire volume of a lot of harvested Spongilla and drying racks are used to keep the drying Spongilla off the ground (FIG. 9). These racks are at least 25cm off the ground and sufficient in number to accommodate the amount of harvested Spongilla. The racks are placed such that walkways are created to allow access to place, turn and collect the sponges without contamination to the sponges. The racks permit air movement above and below the sponges to facilitate drying. The racks are cleaned and disinfected before and after each use. Once dried, the sponges are placed in clean fiber bags. In no event is the Spongilla permitted to come into contact with the ground. The use of home yard drying is strictly prohibited.

Alternatively, commercial scale dryers used to dehydrate foods and pharmaceuticals can be used as appropriate. However, sponge harvesting is generally done in remote rural regions due to the difficulties associated with developing and sustaining artificial “sponge farm” habitats. Consequently, large commercial drying facilities are seldom available. As another alternative, collected sponge colonies can be sent to the repository, a low-temperature storage facility for their quarantine, delayed processing and further investigation.

Drying of Spongilla raw material (the sponges themselves) commences on the day of harvest and is monitored on a daily lot-by-lot basis.

When dried under ambient, open-air conditions temperature, dew point, relative humidity and forecasted precipitation must be closely monitored. Drying time for each lot of harvested Spongilla species is approximately 21 days from the date of harvest of each lot. In certain embodiments, the drying time is about 14-28 days, about 16-26 days, about 18-24 days, or about 20-22 days. In other embodiments, drying time is no more than about 18 days, no more than about 20 days, no more than about 21 days, no more than about 22 days, or no more than about 24 days.

In the event of precipitation or strong wind, the harvested Spongilla is covered with plastic sheeting during the duration of the precipitation or wind, and the drying time extended by the same number of days as the Spongilla was covered. If the ambient air temperature is too low the sponge mass is dried indoors where temperature and humidity can be controlled. It is not essential that a precise temperature or humidity range be maintained, however, the sponge mass should be maintained within a temperature and humidity range suitable for an uninterrupted evaporative process to proceed. For example, temperatures should be above about 60° F. and relative humidity should be below about 90%. In certain embodiments, the temperature is above about 55° F., above about 65° F., above about 70° F., above about 75° F., or above about 80° F. In other embodiments, the temperature is less than about 90° F., less than about 85° F., less than about 80° F., less than about 75° F., or less than about 70° F. In other embodiments, the relative humidity is less than about 85%, less than about 80%, less than about 75%, less than about 70%, less than about 65%, less than about 60%, less than about 55%, or less than about 50%.

The sponge mass is protected from exposure to atmospheric precipitation and excessive temperatures after collection. The sponge mass is dried until residual moisture content is less than about 10%, less than about 9%, less than about 8%, less than about 7%, less than about 6%, less than about 5%, less than about 4%, less than about 3%, less than about 2%, or less than about 1%. If the raw material is to be stored for protracted periods before further processing, residual moisture can be as low as about 0.1% or less. In other embodiments, the residual moisture is less than about 0.2%, less than about 0.3%, less than about 0.4%, less than about 0.5%, less than about 0.6%, less than about 0.7%, less than about 0.8%, or less than about 0.9%. Residual moisture measurements are performed using methods commonly known in the arts of food sciences, analytical chemistry or the pharmaceutical sciences. For example, 10 grams of dried material is placed on a tared weighing boat and then weighed. The weighed material is then exposed to a heat source such as a drying oven or heat lamp operated at a temperature sufficient to evaporate any remaining free or loosely bound water (non-chemically bound). The sample is then cooled in a desiccated chamber and re-weighed. Residual moisture is calculated as the percent difference between the sample weight before drying and the weight after cooling.

The desiccated sponge is processed and sealed in storage bags immediately after the end of the desiccation period in order to minimize exposure to heat, light, moisture, humidity and environmental contamination, which might lead to sponge biometabolites degeneration.

The sponge raw material is maintained under quarantine conditions until routine quality control processes are conduced on the dried sponge material consistent with Good Manufacturing Practice Requirements (GMP) and International Standards Organization (ISO) regiments applicable to food, drugs and cosmetics before being released from quarantine and processed further. Testing includes, but is not limited to, microbiological culturing for pathogens, coliform organisms, bioburden, chemical analysis, taxonomical confirmation, and bioactivity analyses.

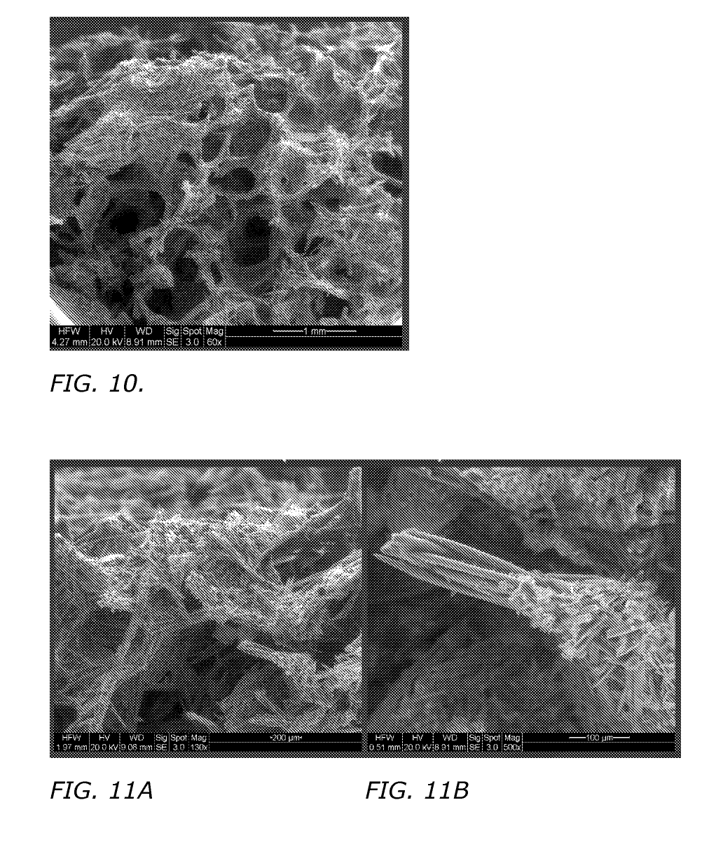

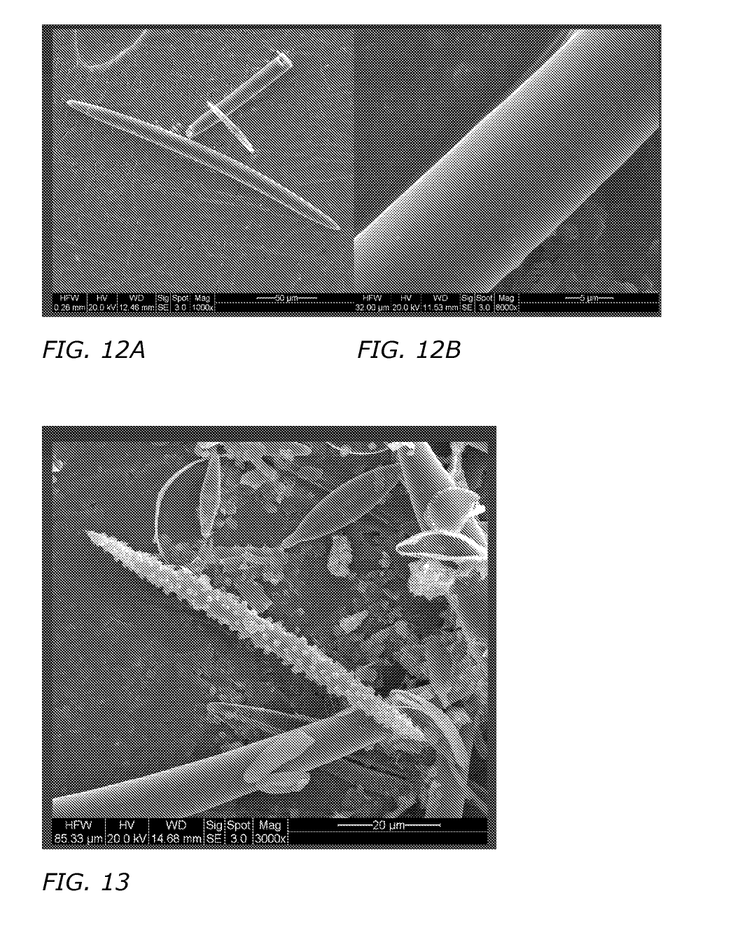

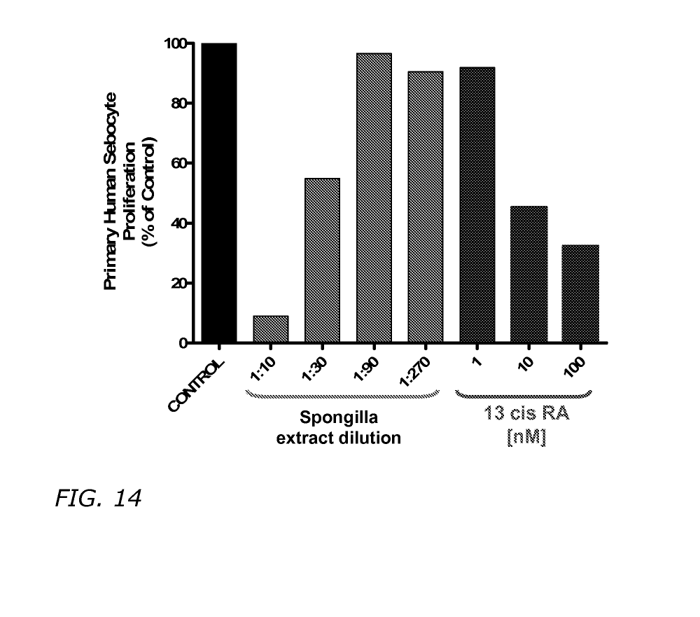

The identity of sponges is easily verified by taxonomic examination of a sponge sample by competently trained marine biologist possessing ordinary skill in the art. For example, surface of Spongilla lacustris specimen from the Astrakhan region is macroscopically very conulose (projecting ectoderm). A scanning electron microscope (SEM) reveals the ectoderm to be hispid (spicules projecting through surface), spicular brushes/small palisades observed, very little spongin (FIGS. 10 and 11). Megascleres (spicules) of S. lacustris consist of oxeas only, which are smooth (FIG. 12). Microscleres (spicules) have tiny spines (FIG. 13). Both types of spicules are slightly curved, uniform size, tips acerate-to-blunt, which is consistent with S. lacustris (shape and distribution on acanthoxea). Presence or absence of gemmules in observed specimen will confirm the stage of sponge. Presence of gemmules in a sample will be consistent with S. lacustris in non-reproductive “resting” stage, which usually takes place in the Astrakhan region late in fall.

Before the material is released from quarantine, each lot is tested and has to comply with applicable regulatory requirements. At minimum each lot has to comply with the Certificate of Analysis (COA) in Table 3.

TABLE 3

| | ACCEPTABLE LIMITS |
|---|---|
| ANALYTE TESTED | (not to exceed) |
| Heavy Metals | |
| Lead, mg/kg | not more than about 1.0 mg/kg |
| Arsenic, mg/kg | not more than about 1.0 mg/kg |
| Cadmium | not more than about 0.2 mg/kg |
| Mercury | not more than about 0.5 mg/kg |
| Radionuclides: | |
| Caesium-137, Becquerel/kg | not more than about 200 Bq/kg |
| (Bq/kg) | |
| Strontium-90, Bq/kg | not more than about 400 Bq/kg |
| Microbiology | |
| Total Colony Count | Not more than about 5 × 10³ |
| (Aerobic and Anaerobic) | Colony forming units (CPU) |
| Coli forms | Absent |
| Salmonella | Absent |
| Yeast and Mold | Absent |
| Miscellaneous | |
| Residual Moisture | Not to exceed about 10% |

In addition, each COA includes a signed statement of a qualified aquatic biologist attesting to the taxonomic identity of the sponge and conforming to specifications of HPLC, UV absorption, pH and dry weight recovery analyses for quality control and batch-to-batch consistency.

After release from quarantine, the raw, dried sponge material is further refined and processed to a standard particle size using sieves. All processing after the initial drying phase is conducted in environmentally controlled facilities that comply with GMP and ISO guidelines. Manufacturing personnel are trained in GMP, ISO and MSDS (a Material Safety Data Sheet) procedures and all manufacturing process closely monitored and recorded.

Personnel working with Spongilla powder exercise appropriate precautions to prevent opportunities for inhalation of Spongilla powder dust, and to prevent direct contact with skin, mucous membranes and eyes. Personnel wear personal protective clothes and protective devices such as trousers, jacket, shoes, caps, gloves, dust respirators, tightly fitted goggles.

In order to properly maintain the equipment and rooms, a regular cleaning and disinfection schedule for the mill and milling room (not less than once each day during processing) is established and maintained. All additional equipment or tools used is also cleaned and disinfected daily during the processing to eliminate contamination. Each cleaning is documented in a logbook.

The dried sponge is extremely fragile and requires only slight, gentle grinding to form a consistently fine particulate. The dried sponges collected and processed in accordance with the present disclosure should not be processed using aggressive grinding techniques, rather the dried sponge is processed gently to avoid crushing debris that may be present in the sample. For example, shells from aquatic mollusks may contaminate the crude sample; grinding of the crude sponge preparation should be conducted in a fashion that will not pulverize the contaminating shells to a degree that they would not be removed in the sieving processes. Several grinding and sieving steps are performed to reduce average particle size to no more than about 0.2 mm. First a course grind and sieving process is used to reduce particle size to at least about 2 mm. This initial sieving process also permits visual inspection and removal of remaining non-sponge debris and is followed by subsequent grinding and sieving processes where the raw material is ultimately reduced to no more than about 0.2 mm particles.

Next the sized material is ground and sieved again to reduce particle size to no more than about 0.2 mm. After the desiccated sponge powder is ground, it is further purified and separated from contaminants by processing the powder with sieves having progressively smaller apertures (about 1 mm, about 0.5 mm, and about 0.2 mm respectively). All processing is conducted under GMP conditions.

The milling and storage room is equipped with a ventilation system of potent exhaust fans or other engineering controls to avoid airborne dust. The desiccated sponge material is processed and sealed in the storage bags immediately the same day) after the desiccation period in order to minimize exposure to heat, light, moisture, humidity and environmental contamination, which can lead to degradation of the processed sponge material.

After final grinding and sizing processes are completed, the dried sponge material is immediately packaged in bulk (5-10 kg) in airtight moisture-proof storage bags, which protect the processed material from contamination, direct light and humidity. All air is removed from each bag before it is sealed. The storage bags are durable and resistant to breaking, tearing and moisture. It is recommended that sealed storage bags are placed in an outer bag or container composed of thick moisture-resistant plastic. At least ten 100-gram samples of the processed material from each annual harvest are collected and retained for initial quality control purposes and for long-term sample retention.

The storage bags, outer bags and containers are labeled with product description (i.e., Desiccated Spongilla), formulation (i.e., Powder), particle size, lot number, weight, date harvested, date processed and packaged, storage conditions to be observed (i.e., cool, dry and dark) and the name of the manufacturer.

Each processed lot of Spongilla is tested in accordance with sound scientific principles and, where applicable, GMP protocols. Each processed lot of Spongilla includes a Certificate of Analysis (COA) signed by a laboratory conducting the testing and attesting to processed Spongilla material's composition, purity, and activity. The COA provides analytical test results and a statement from the testing facility's laboratory that the lot tested complies with applicable regulatory requirements. Moreover, each COA includes a signed statement of a qualified aquatic biologist attesting to the taxonomic identity of the sponge. The processing and certified Spongilla material is referred to herein as “Spongilla powder.”

Spongilla powder collected, processed and stored in accordance with the disclosed teachings is stable for at least 3.5 years if stored in the dry dark conditions at a temperature not higher than 80° F. In additionally embodiments, the Spongilla powder is stable for a minimum of four years, five years, or six years. Additionally, if the bulk Spongilla powder is stored below 32° F., the powder is stable for more than five years, more than six years, more than eight years, more than 10 years, more than 12 years, or more than 14 years.

As use herein, quality control refers to evaluation of the physical, chemical, and biological activity of several lots of Spongilla powder. Specifically the quality control of Spongilla powder comprises HPLC, UV absorption, pH, dry weight recovery analysis, and disease-specific biological assays of Spongilla powder extracts.

The therapeutic compositions disclosed herein comprise from approximately 0.1% to approximately 100% substantially pure Spongilla powder. In other embodiments, the therapeutic compositions comprise approximately 1-99%, approximately 10-90%, approximately 20-90%, approximately 30-90%, approximately 40-90%, approximately 50-90%, or approximately 60-90% substantially pure Spongilla powder. In other embodiments, the compositions comprise at least about 10%, at least about 15%, at least about 20%, at least about 25%, at least about 30%, at least about 35%, at least about 40%, at least about 45 at least about 50%, at least about 55%, at least about 60%, at least about 65%, at least about 70%, at least about 75 at least about 80%, at least about 85%, at least about 90%, at least about 92%, at least about 95%, or at least about 97% substantially pure Spongilla powder.

The compositions can be optionally compounded with pharmaceutical excipients including, but not limited to water, saline, buffered phosphate, oils, gels, waxes, emollients, glycerin, cleansers, fragrances, colorings, antiseptics and anesthetics. Suitable waters include water for injection, irrigation water, distilled water, deionized water, and floral water among others. Even clean tap water is acceptable for some applications. The concentrations of the aforementioned excipients can range from 0.001% to 50% or more depending on the requirements and at the discursion of the formulation scientist, pharmacist or prescribing physician. Such ranges are well known in the art and can be determined without undue experimentation. Other excipients that may be used in accordance with the teachings of the present invention may include from approximately 0.1% to 25% coral powder, from approximately 0.1% to 25% seaweed powder, from approximately 0.1% to 10% hydrogen peroxide and from approximately 0.1 to 10% of an inorganic or organic acid such as, but not limited to boric acid, hydrochloric, ascorbic acid, salicylic acid, and others.

The therapeutic compositions of the present invention generally comprise from 0.8 to 1.5 grams of substantially pure Spongilla powder, and at least one additional excipient selected from the group consisting of from 0.1 to 0.5 grams of green seaweed powder, from 0.1 to 0.5 grams of white seaweed powder, from 0.1 to 0.5 grams of coral powder, from 0.1 to 0.5 grams of Plantain powder, from 0.5 mL to 5 mL of 0.1% to 10% hydrogen peroxide, from 0.5 mL to 5 mL of 0.1% to 10% boric acid and from 0.5 to 5 mL of water, from 0.5 mL to 5 mL enzyme gel (comprising water, hydroxyethylcellulose, hyaluronic acid, propylene glycol, methylparaben, tetrasodium EDTA and propylparaben in proportions suitable for topical applications as known to those skilled in the art), from 0.5 mL to 10 mL jojoba oil. Other excipients such as, but not limited to saline, buffered phosphate, oils, waxes, emollients, glycerin, cleansers, fragrances, colorings, antiseptics and anesthetics may be added as desired or required.

### EXAMPLES

### Example 1

### Quality Control of Spongilla Harvested for Formulation of Spongilla-Based Therapeutic Compositions for Treating and Preventing Acne

In this embodiment, the quality control used for the manufacturing of the Spongilla-based therapeutic compositions for the treatment of acne is described. Extracts of several lots of interest were generated. Because these extracts were tested for the efficacy of the treatment of acne it was necessary to determine the variation among them, whether it is on daily basis or annual.

Spongilla test materials were extracted with ethanol, and then tested for their physical and chemical behavior under various stringent protocols. They included HPLC, UV absorption, pH, and dry weight recovery after 40% ethanol extraction.

Preparation of Extracts. Dried Spongilla powder material (0.2 g) was suspended in 1.0 ml in 40% reagent grade ethanol and mixed by rocking for 10 min at room temperature. The extracts were then separated from the extracted material by centrifugation (10,000×g) for 10 min. The volume of the recovered extract was adjusted back to 1.0 ml and 0.2 g of fresh Spongilla powder material added. The extraction process was repeated for a total of 15 times. Extracts were clarified by passing though nylon filters containing 0.2 μm pores.

Dry Weight Determination. 500 μl of each extract was delivered to a tared glass flask and dried down at 37° C. under vacuum using a roto-vap apparatus. The flasks containing the dried material were then re-weighed.

pH Determination. Each dried extract was dissolved in 500 μl of water. The pH was determined using an Orion combination pH probe.

UV Spectrophotmetric Analysis. The absorbance at 200-600 nm of each extract, diluted 1/100 in 1.0 ml of water, was determined using a Beckman DU640B spectrophotometer.

Reversed-Phase C18 HPLC Analysis and Fractionation.
Sample: Spongilla extracts
Sample Preparation: Filtered on Whatman nylon filt.disc 0.45 micrometer
Injected: 5 microliter
Buffers: A: 0.1% TFA-Water(v/v)
   B: 0.1% TFA-60% acetonitrile-water (v/v)
Column: Vydac C18 (218TP54), 4.6×250 mm 15-20 micron particle size,
   300 Angstrom pore size
Detection: 210 nm and 254 nm
Flow: 1.2 mL/min
Gradient: 0% B for 10 minutes, 0-100% B in 100 minutes.
Temperature: Room Temperature

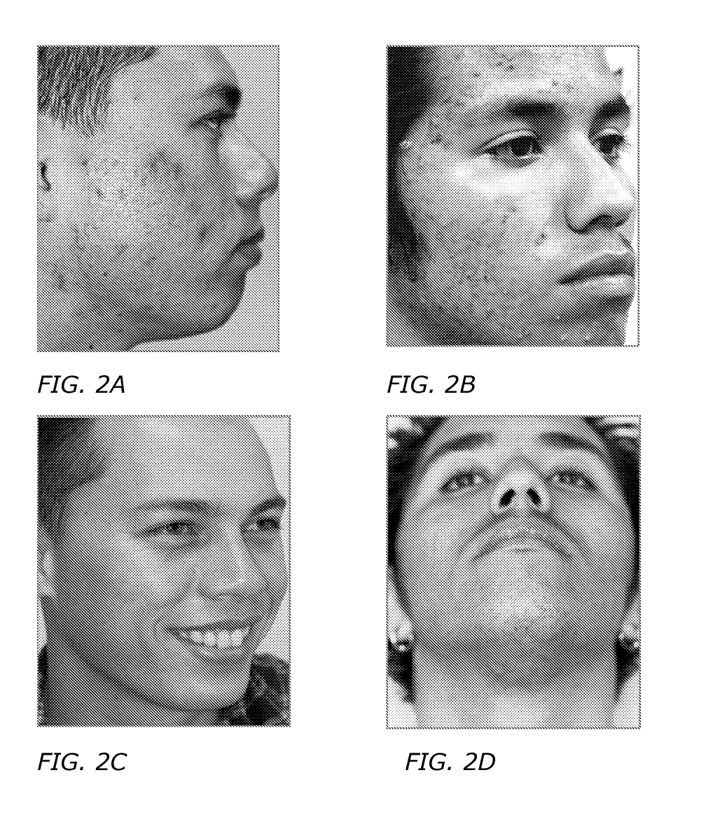

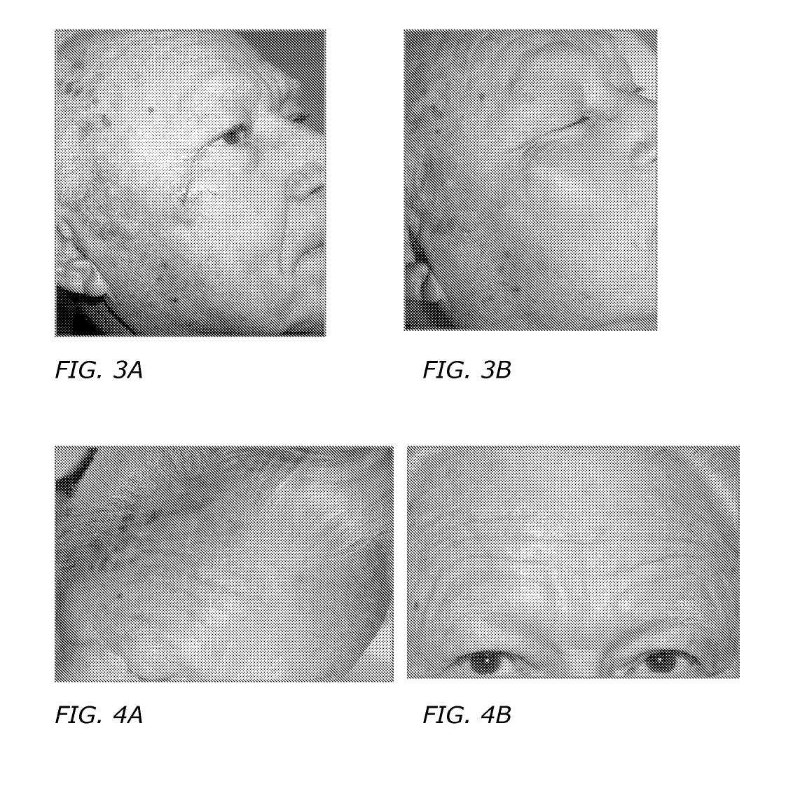

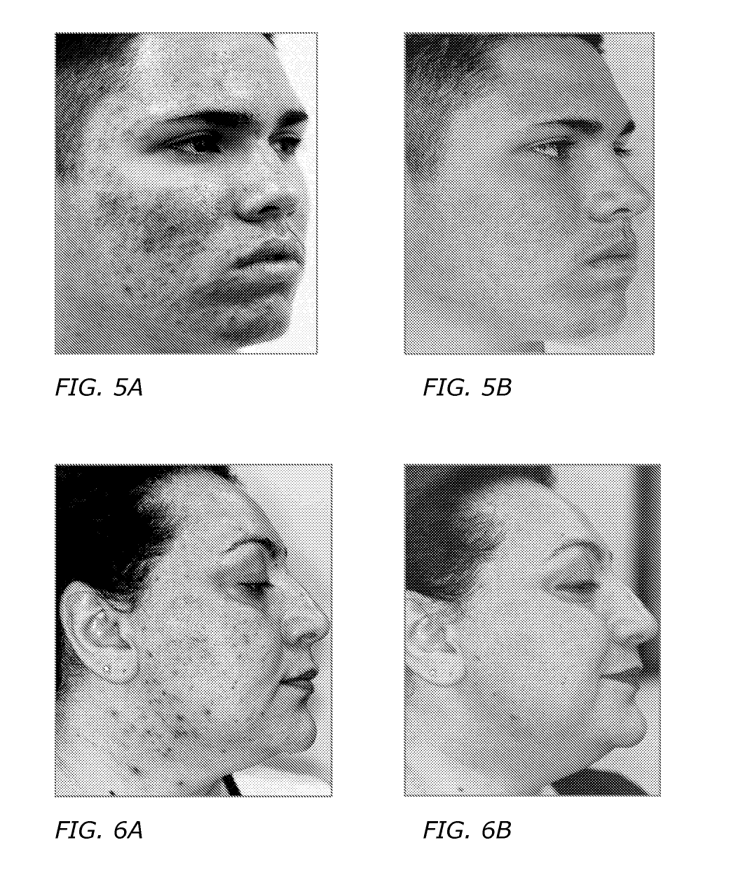

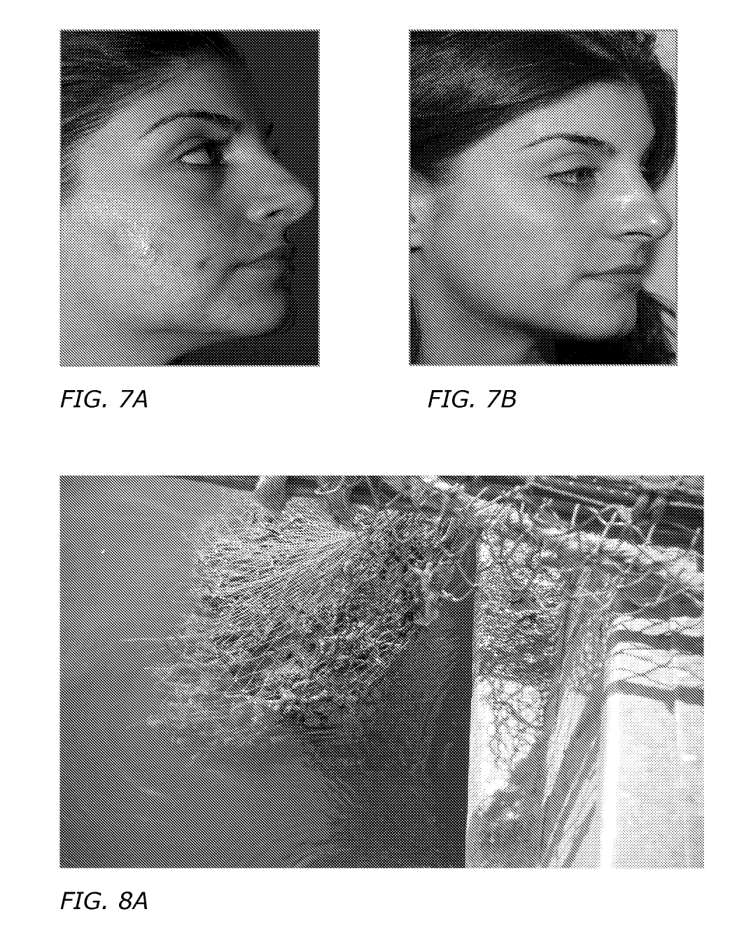

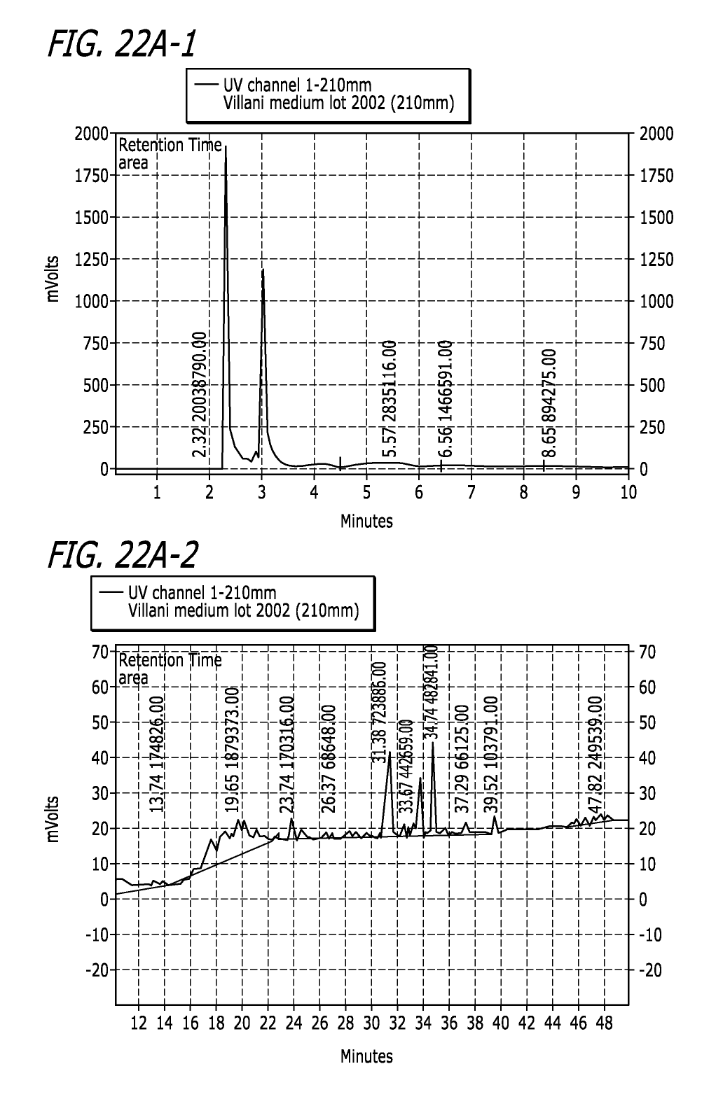

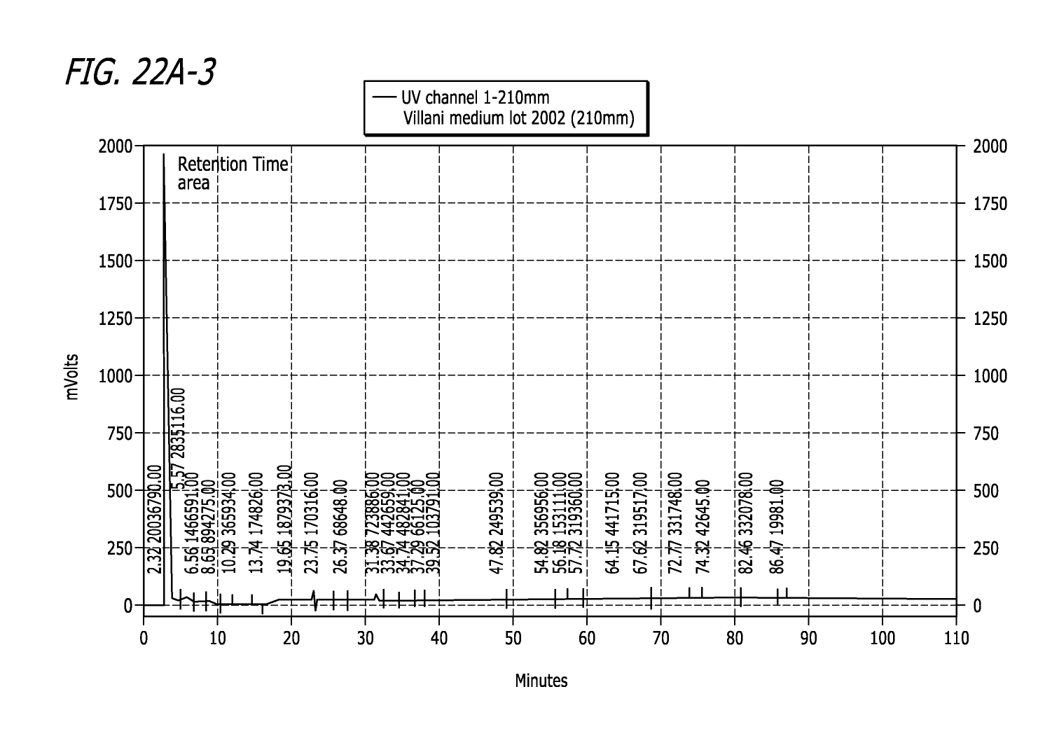

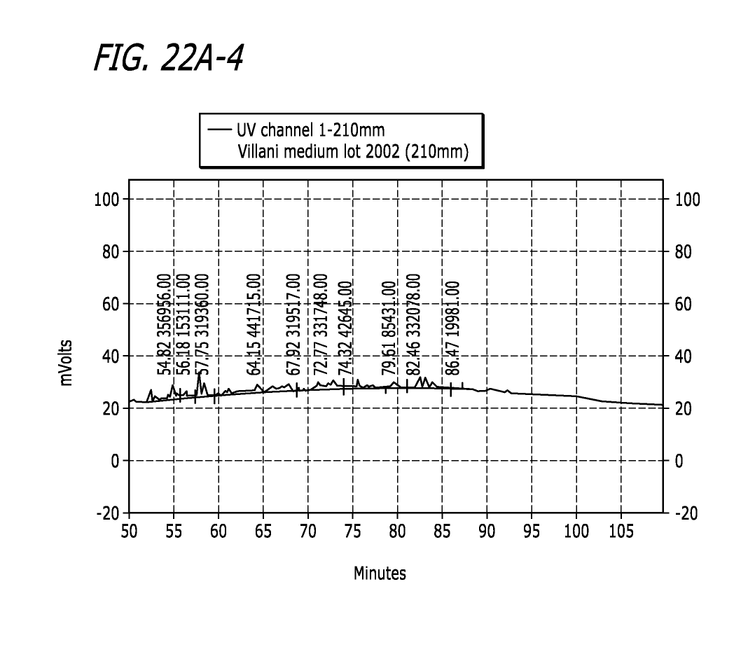

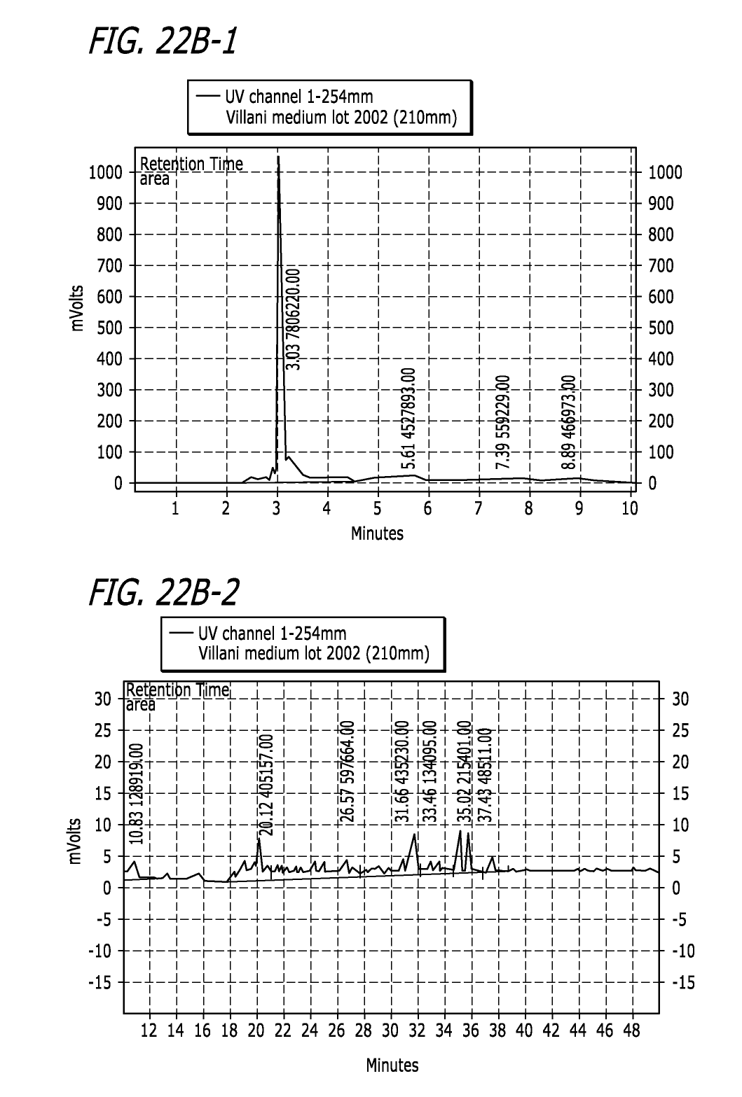

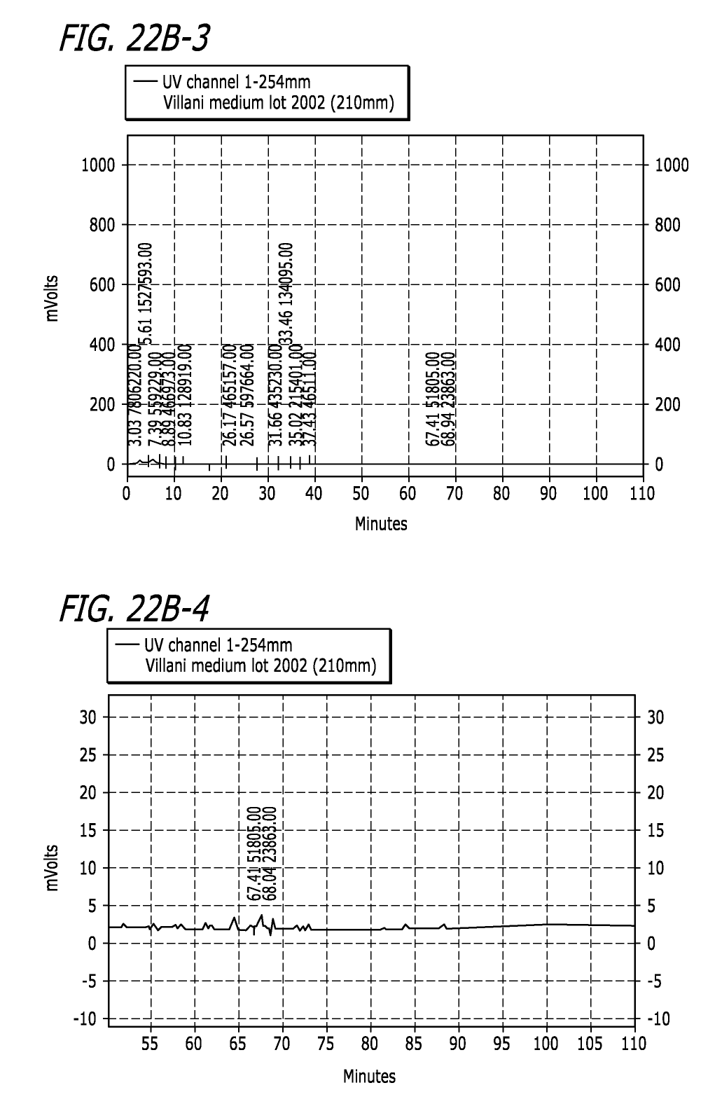

The UV absorption profiles at 210 and 254 nM of the fractionated material were generated (FIGS. 22A-1-4 and 22B-1-4).

Generation of Primary Sebocytes. Human sebaceous glands were dissected from facial skin obtained from cosmetic surgeries and placed onto a confluent monolayer of NIH 3T3 fibroblasts that had been growth arrested by treatment with mitomycin C. Dissections were carried out over 8 hr and generated approximately 20 glands. Out-growth from the glands occurred within one week of culture. By 2.5 weeks, the culture generated sufficient number of sebocytes (present in a monolayer of tightly packed raised cells exhibiting a cobblestone morphology) for the MTT assay. By this time, the glands and the primary sebocytes immediately adjacent to the glands exhibited marked involution, indicating that primary sebocytes exhibit a finite life-span in culture.

The primary sebocytes were detached for sub-culture by a series of increasingly severe treatments designed to determine the gentlest means by which to first remove the fibroblast feeder cells and then dissociate the sebocytes from the culture flask and from themselves. It was determined that the former is effectively accomplished by treating the culture with 0.25% trypsin/0.02% EDTA (TE) for 7 min at 37° C. The activity of the trypsin was then neutralized by the addition of media containing 10% fetal calf sera, the fibroblasts were removed, and the culture flask washed once with DPBS. The remaining sebocytes were detached through additional rounds of treatment with TE. In each round, the proteolytic activity of the trypsin was neutralized, the dislodged sebocytes removed and the remaining cells washed with DPBS. After 3 rounds, the pooled sebocytes were centrifuged, counted and resuspended at a concentration of 1×10⁵/ml. A total of ˜800,000 cells were recovered from the primary culture flask. Two hundred microliter aliquots (20,000 cells) were placed in each well of 96 well culture plate and the cells allowed to attach for 24 h in culture. In addition, cells were also plated into a T25 culture flask for subculturing.

Human primary sebocytes were incubated with different concentrations of lots #1-10 for two days. After this, the cells were seeded at 50,000 cells/well of a 96 well plate. After culture overnight to allow adhesion, the media was buffered to 50 mM HEPES, pH 7.4 by the addition of a 1/10 volume of 0.5 M HEPES. Extracts of different lots of Spongilla powder extracts were added at the desired concentrations to quadruplicate wells and the plate cultured for 2 days. The plate was then centrifuged at 1200 rpm for 10 min and the culture supernatants removed. Two hundred μl of complete media containing 0.86 mg/ml MTT was added to each well and the plates cultured for an additional 4 h. The plates were centrifuged again, the media removed and the well contents solubilized in 100% DMSO. The optical density at 560 nm of each well was determined and the averaged mean of the duplicate wells calculated. The background OD560 value, determined from control wells receiving media containing no MTT, was subtracted from these values and the data normalized to that of the control wells receiving equivalent volumes of water.

Study of Inhibition of Bacterial Growth. P. acnes strain 11828 was obtained from ATCC and grown under anaerobic conditions in DIFCO™ Reinforced Clostridial Medium supplemented with 0.5 g/L sodium thioglycollate. Different dilutions of Sterile Spongilla extract were added to the liquid culture and bacterial growth was monitored by reading the OD (625 M) of the bacterial culture.

Results

Dry Weight and pH value determinations. Extracts of all lots received were dried and a sample containing 500 μl was weighed. The weights of these lots ranged between 5-7 mg, with the exception of lot #2 (Medium 2002 B) which was 3.5 mg/500 μl and lot #10 (Coarse 2004 D) which was 10.6 mg/500 μl. The pH values of all lots were very comparable and ranged between 5.26-5.78, with the exception of lot #10 (coarse 2004 D) which was 6.61 (Table 1).

TABLE 4
Dry weight and pH values of ten lots of test articles.

| | DRY WEIGHT | |
|---|---|---|
| SAMPLE | (500 μL) | pH |
| 1. Medium 2002 A | 5.0 Mg | 5.26 |
| 2. Medium 2002 B | 3.5 Mg | 5.42 |
| 3. Coarse 2002 C | 6.9 Mg | 5.42 |
| 4. Fine 2003 C | 6.1 Mg | 5.54 |
| 5. Medium 2003 A | 5.0 Mg | 5.62 |
| 6. Medium 2003 B | 5.3 Mg | 5.78 |
| 7. Medium 2004 A | 6.4 Mg | 5.65 |
| 8. Medium 2004 B | 6.5 Mg | 5.50 |
| 9. Medium 2004 C | 5.2 Mg | 5.38 |
| 10. Coarse 2004 D | 10.6 Mg | 6.61 |

UV Absorption. The UV absorbance at 200-600 nm of the different lots was then determined. All ten lots showed similar UV absorption profile with the exception of lot #2 which had an absent peak at about 200-222 nm, lot #7 which shifted to the right at 200-250 nm and lot #10 which showed a high peak at UV 200-250 nM.

HPLC Analysis. Two different wavelengths were employed to determine the HPLC profiles of the various lots. These were done at 210 nm and 256 nm. A comparison of the various lots under these circumstances is described below:

At 210 nm absorption, Lots #1, 2, and 3 have almost similar profile when the gradients were examined after 0-10, 10-50, 50-110 and 0-100 min run. Lots #4, 5, 6, and 7 showed higher peaks at 17.5-25 min when compared to lots #1, 2 and 3. Lots #6, 7, and 8 have slightly higher peaks at 31-36 min. Lot #10 has a different profile at 10-50 min post run with many peaks found during this time period that were not observed with other lots. However, many of the peaks found in other lots between 50-106 min run disappeared from lot #10.

At 254 nm absorption, Lots #1, 2, and 3 have almost similar profile when the gradients were examined after 0-10, 10-50, 50-110 and 0-100 min run. Lot #4 showed a similar profile to lots #1, 2 and 3, but had a higher peak at 19.63 min. Lot #5 showed a similar profile to lots #1, 2, 3 and 4 with the exception that it had a peak at 96-106 min post run. Lots #6 and 7 also showed similar profiles to the other lots except that they have no peaks at later time points (80-110 min). Lots #8, 9, and 10 showed almost similar profile to other lots, but lot #9 had a higher peak than the other lots at 31.3 and 35.47 min.

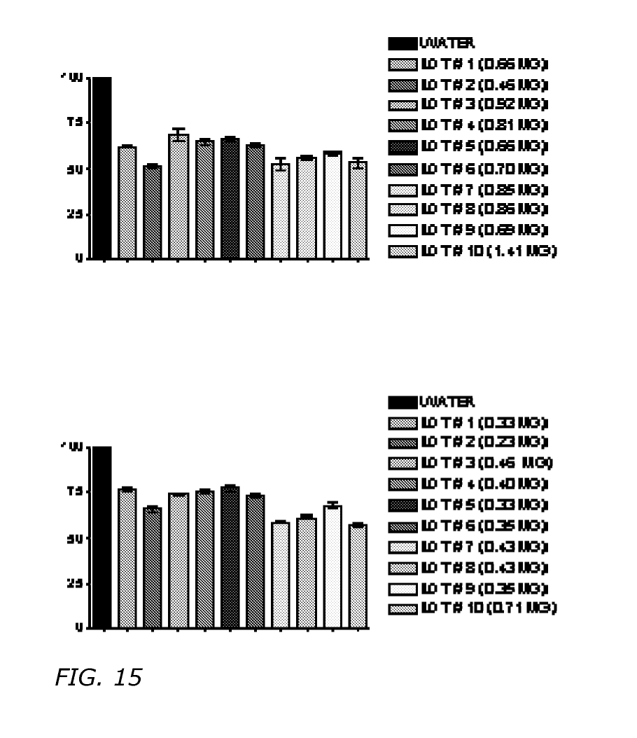

Effect on Sebocyte Proliferation. After determining the chemical properties of the different lots, the effects of these extracts on sebocytes proliferation were examined. The results in FIG. 15 demonstrate that the lots significantly inhibited sebocytes proliferation to almost similar extent, at the desired concentrations utilized. Although different concentrations of these extracts were used, they were similarly effective. The significant values of inhibition are shown in Table 5:

TABLE 5

| | Concentration | Concentration |
|---|---|---|
| | (Value of Inhibition) | (Value of Inhibition) |
| Lot # 1 | 0.66 mg/ml (P < 0.0003) | 0.33 mg/ml (P < 0.0038) |
| Lot # 2 | 0.46 mg/ml (P < 0.0008) | 0.23 mg/ml (P < 0.0027) |
| Lot # 3 | 0.92 mg/ml (P < 0.010) | 0.46 mg/ml (P < 0.003) |
| Lot # 4 | 0.81 mg/ml (P < 0.0016) | 0.40 mg/ml (P < 0.0007) |
| Lot # 5 | 0.66 mg/ml (P < 0.009) | 0.33 mg/ml (P < 0.0053) |
| Lot # 6 | 0.7 mg/ml (P < 0.0007) | 0.35 mg/ml (P < 0.0028) |
| Lot # 7 | 0.85 mg/ml (P < 0.0053) | 0.43 mg/ml (P < 0.0004) |
| Lot # 8 | 0.86 mg/ml (P < 0.001) | 0.43 mg/ml (P < 0.0003) |
| Lot # 9 | 0.69 mg/ml (P < 0.0006) | 0.35 mg/ml (P < 0.0019) |
| Lot # 10 | 1.41 mg/ml (P < 0.0034) | 0.71 mg/ml (P < 0.0014) |

All lots examined (#1-10) significantly inhibit sebocytes' proliferation when the Spongilla extracts were examined at the indicated concentrations in the MTT assay.

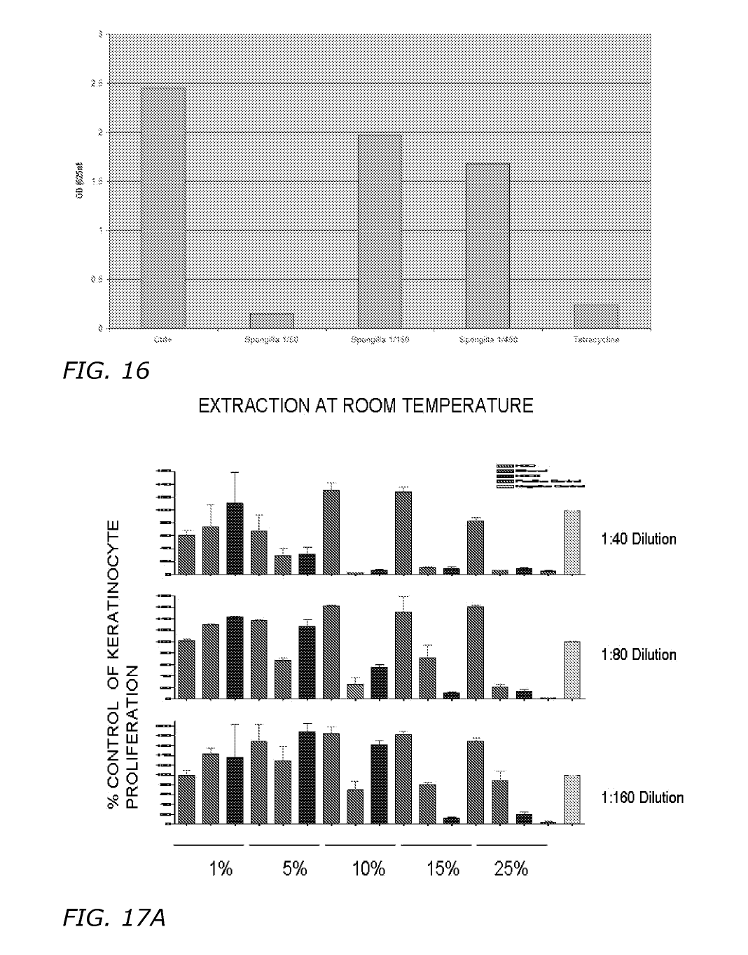

Bacterial growth inhibition. All lots examined (#1-10) significantly inhibit P. acnes growth in the presence of Spongilla extract (prepared in the optimized conditions; 15% wt/vol in H₂O₂) and tetracycline (FIG. 16)). Spongilla extracts (1:50) or 1:150 dilutions were incubated with P. acnes. Tetracyclin was used as a positive control the growth of bacteria was then examined. Bacterial growth is greatly inhibited at the highest concentration of Spongilla extract tested (1/50) and moderately inhibited by the two other concentrations tested (1/150 and 1/450).

Very small differences were observed in the physical and chemical profiles among the 10 lots of the test articles.

Lot #10 had a higher dry weight and higher pH than the rest of the lots. Also, lot #10 had a different HPLC profile at 10-50 min run with many more peaks found at this gradients run when compared to the other lots. In contrast many of the peaks found in other lots between 50-106 min were not observed in lot #10.

Although the HPLC profile of lot #2 was similar to lots 1 and 3 when examined under two wavelengths (210 and 254 nm), lot #2 showed a different UV absorption profile. Further, lot #2 has a lower dry weight when compared to other lots.

All 10 lots behave similarly in inhibiting sebocyte proliferation and P. acnes growth.

Even though multiple physical and chemical similarities among the 10 lots were present and all 10 lots behave similarly in inhibiting sebocyte proliferation and P. acnes growth, it was unknown what caused a different UV absorption profile in Lot #2 and why the peaks found in other lots between 50-106 min were not observed in lot #10. Because lot-to-lot consistency is critical for safety and efficacy of Spongilla-based therapeutic compositions for human use, Lots #2 and 10 were rejected for the purpose of manufacturing of any Spongilla-based therapeutic compositions; and Lots #1, 3, 4, 5, 6, 7, 8, and 9 were accepted for the purpose of manufacturing of Spongilla-based therapeutic compositions for treating and preventing acne.

### Example 2

Optimization of the Extraction Method of Spongilla Powder for Inhibition of Primary Human Keratinocytes

Spongilla extracts that were prepared using different solvents and at different conditions do not possess the same biological activity. It is therefore, crucial to determine the best conditions of extraction to retain and maximize the activity of interest.

Epidermal hyperplasia—excessive keratinocyte proliferation leading to expansion of the epidermis in association with epidermal shedding—is the major manifestation of psoriasis.

Epidermal hyperplasia also occurs under physiological conditions (i.e., during wound-healing) and is a consequence in many individuals of topical treatment with all-trans retinoic acid (RA) or its precursor, all-trans retinol. Epidermal hyperplasia and normal epidermal growth are, to some degree, at least, differentially regulated. The Spongilla-based topical therapeutics interfere with hyperplastic epidermal growth without disturbing normal skin physiology.

Spongilla test materials were extracted with three different solvents (water, 40% ethanol or 3% hydrogen peroxide). Different extraction times, number of cycles and amount of test materials added per cycle were tested. The in vitro activity of the extracts was evaluated for its capacity to kill a keratinocyte cell line in an MTT assay.

Preparation of Extracts. Spongilla powder was suspended in either water, 40% reagent grade ethanol or 3% H₂O₂ and mixed by rocking for different periods of times at room temperature or 60° C. The extracts were then separated from the extracted material by centrifugation (10,000×g) for 10 min. When the protocol required more than one cycle of extraction, a second cycle was initiated by adjusting the volume of the recovered extract back to the starting volume and adding fresh material. After the last cycle, the final extract was clarified by passing through nylon filters containing 0.2 μm pores. The extract was then dried and resuspended in sterile deionized water.

MTT assay. Normal human epidermal keratinocytes (NHEK, Clonetics) were seeded at 20,000 cells/well of a 96 well plate. After that culture was left overnight to allow adhesion, the media was buffered to 50 mM HEPES, pH 7.4 by the addition of a 1/10 volume of 0.5 M HEPES. Serially diluted extracts were added to quadruplicate wells to yield the indicated concentrations and the plates cultured for 2 days. The plates were then centrifuged at 1200 rpm for 10 min and the culture supernatants removed. Two hundred μl of complete media containing 0.86 mg/ml MTT was added to each well and the plates cultured for an additional 4 hr. The plates were centrifuged again, the media removed and the well contents solubilized in 100% DMSO. The optical density at 560 nm of each well was determined and the averaged mean of the duplicate wells calculated. The background OD560 value, determined from control wells receiving media containing no MTT, was subtracted from these values and the data normalized to that of the control wells receiving equivalent volumes of water.

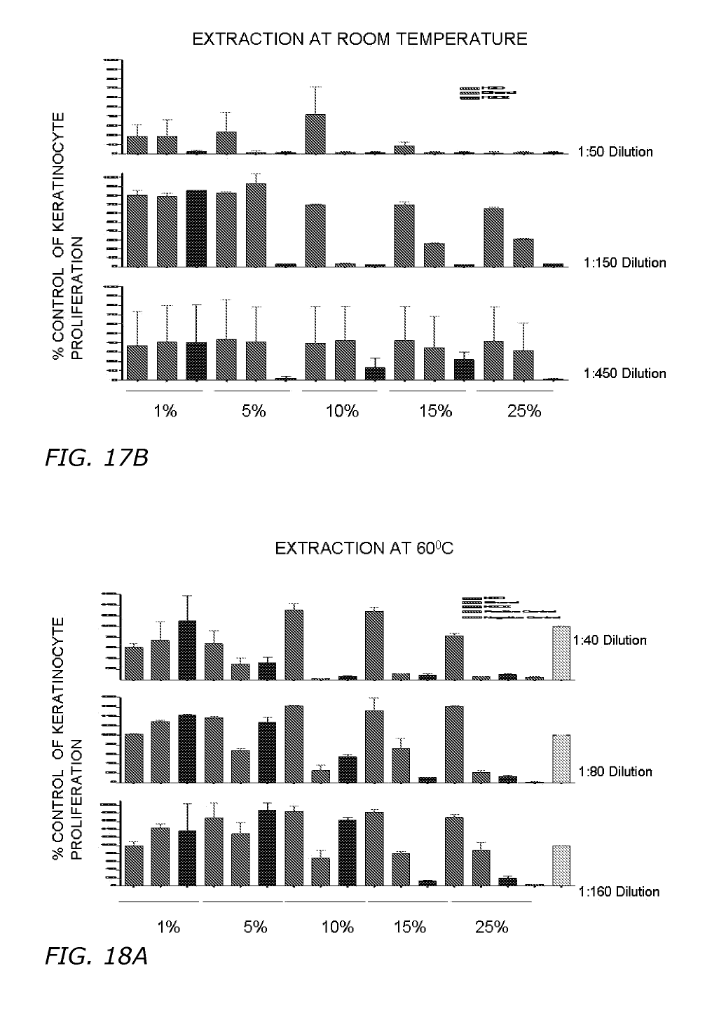

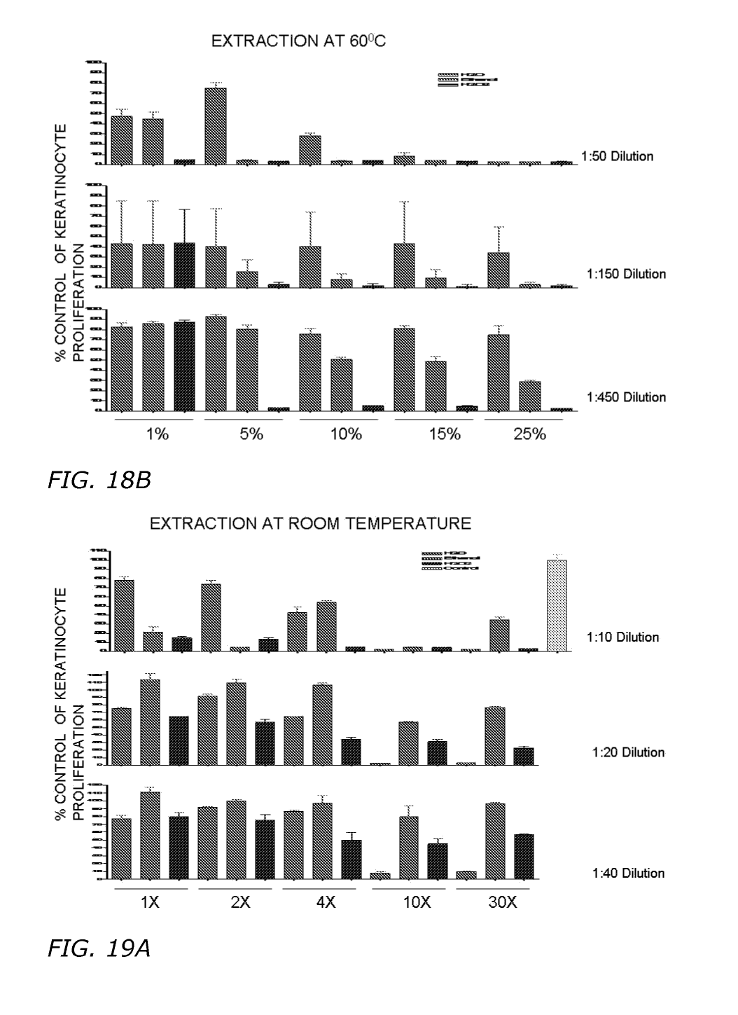

Effect of temperature during extraction on the activity of the extracted materials. Extraction of Spongilla material was conducted at two different temperatures (room temperature (FIG. 17) and 60° C. (FIG. 18)) and the activity of the extracted material was analyzed using the MTT assay on NHEK cells. FIGS. 17 and 18 show the results of three separate experiments for each temperature. Spongilla extracts with good killing activity can be generated when extracted at room temperature or 60° C., but the killing activity of the Spongilla extract is noticeably enhanced when the extraction is performed at 60° C.

Effect of the concentration of Spongilla material in the extract on the final activity of the extract. Extracts with different concentrations of Spongilla material were prepared, ranging from 1% to 25% (weight/volume) and the activity of the resulting extract was tested. FIGS. 17 and 18 show the results. In these experiments three different dilutions of each extract were assayed for their killing potential in the MTT assay. In general, the more concentrated extracts retain more activity even when diluted (for example, compare 25% wt/vol to 5% wt/vol in FIG. 17 and notice that even though there was good killing for the 5% at 1/40 dilution, there was no killing activity when diluted to 1/160 as opposed to the 25% which still kills very well at 1/160 dilution).

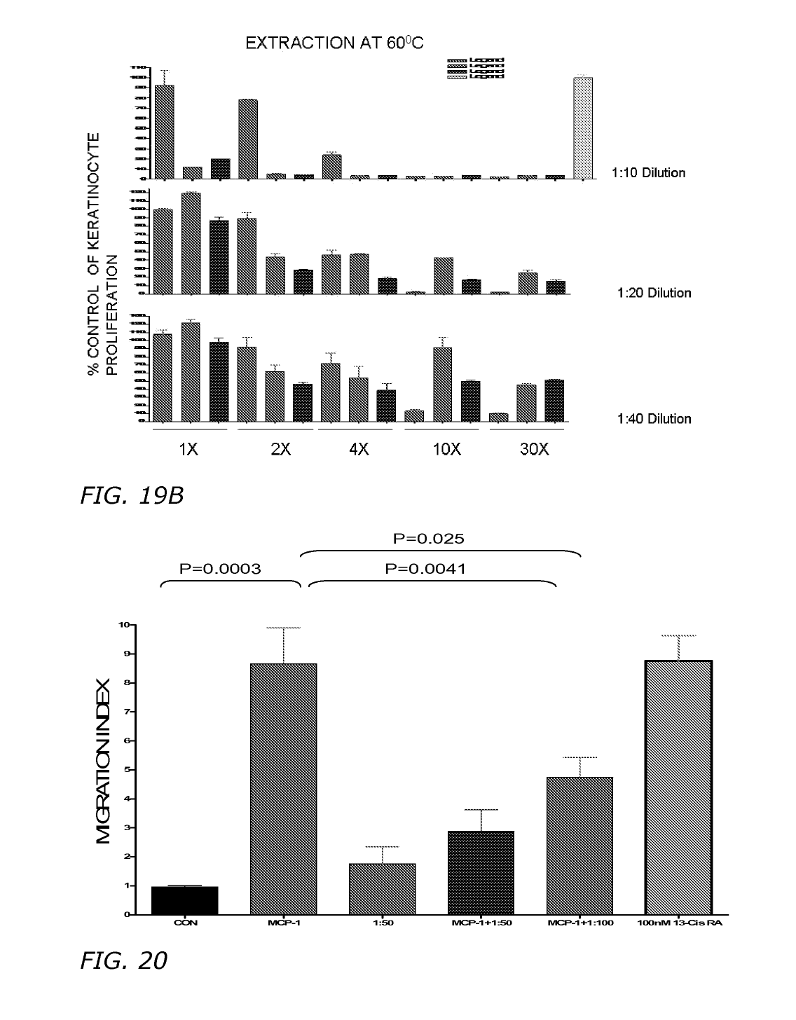

Effect of the solvent used for the extraction of the Spongilla material on the activity of the extracted material. Spongilla extracts made in three different solvents (water, 40% ethanol or 3% H₂O₂) were compared for their killing potential in the MTT assay. Results are shown in FIGS. 17 and 18. The different solvents generate extracts that do not have the same killing potential. FIG. 19 shows experiments using 1× extracts concentrated 15 times (by resuspending the dried extract in a volume 15 times smaller than the original volume). In these experiments, ethanol and H₂O₂ have the best killing profile at 5% wt/vol and above of Spongilla material. FIG. 19 shows a surprising effect of water at higher number of cycles.

Extracts at 5%, 10%, 15% and 25% prepared in ethanol or H₂O₂ at room temperature inhibit cell proliferation when added at 1:40 and 1:80 dilutions (FIG. 17A), or 1:50 and 1:450 dilutions (FIG. 17B) to these cells. A 1:160 dilution of these extracts was less potent, although extract prepared with H₂O₂ are still effective. In the control, 25% alcohol was used as a positive control and no MTT as a negative control. At 1:450 only extract prepared with H₂O₂ is effective. In conclusion, extracts prepared in ethanol or H₂O₂ at room temperature inhibit keratinocyte proliferation and extraction with H₂O₂ is more potent than extraction with ethanol, since even at 1:1450 dilution, H₂O₂ extracts inhibit keratinocyte proliferation. Products generated by extraction with 15% and 25% H₂O also have inhibitory activity for keratinocytes when the extracts are used at 1:50 dilution.

Extracts at 5%, 10%, 15% and 25% prepared in ethanol or H₂O₂ at 60° C. inhibit keratinocyte proliferation when added at 1:40 and 1:80 dilutions (FIG. 18A) and at 1:50 and 1:450 dilutions (FIG. 18B) to these cells. A 1:160 dilution of these extracts is less potent, although extracts prepared with H₂O₂ are still effective. At 1:150 dilution only extracts prepared in ethanol and H₂O₂ but not in H₂O are effective, whereas at 1:450 only the extract prepared with H₂O₂ is inhibitory for keratinocyte proliferation. Extracts prepared in ethanol or H₂O₂ at 5% and above (10%, 15% and 25%) are superior to extracts prepared in H₂O. Extraction with H₂O₂ is more potent than extraction with ethanol, since even at 1:1450 dilution, H₂O₂ extracts inhibit keratinocyte proliferation. Products generated by extraction with 15% and 25% H₂O also have inhibitory activity for keratinocytes when the extracts are used at 1:50 dilution

No differences are observed among extracts prepared at room temperature (FIG. 17) versus those prepared at 60° C. (FIG. 18).

Effect of increasing cycle number on the activity of the final extract. Repeated extraction cycles were performed at 20% wt/vol of Spongilla material. At each cycle fresh material was added to the extract in an effort to concentrate the extract. FIG. 19 shows the results of these studies at two different temperatures (room temperature, FIG. 19A; 60° C., FIG. 19B) and three different dilutions of each extract. First, the killing potential is increased by adding more cycles. Ethanol and H₂O₂ are good solvents even at low number of cycles (dilution 1/10) whereas water extraction did not give good results. Intriguingly, water becomes a very good solvent when the number of cycles is increased to at least 10, even better than ethanol and H₂O₂ (see 1/20 and 1/40 dilutions).

The different extracts were examined at 1:10, 1:20 and 1:40 dilutions for their ability to inhibit keratinocyte proliferation in the MTT assay. 1x and above (2×, 4×, 10× and 30×) concentrations of extracts prepared in ethanol or H₂O₂ are inhibitory for keratinocyte proliferation when used at 1:10 dilution. 10× and 20× of all extracts including those prepared with H₂O are effective when used at 1:10 dilution. Surprisingly 10× and 30× extracts prepared in H₂O are more effective than those prepared in ethanol or H₂O₂ when used at 1:20 and 1:40 dilutions.

Extracts, particularly after several rounds of extractions (10× and 30×) prepared in H₂O, ethanol or H₂O₂ at room temperature (FIG. 19A) are inhibitory for keratinocyte proliferation, when used at 1:10 dilution in the MTT assay. 1× and 2× extracts made by H₂O₂ or ethanol, but not by H₂O are inhibitory when used at 1:10 dilution. 10× and 30× extracts made by H₂O are more potent than those made by H₂O₂ or ethanol when used at 1:20 and 1:40 dilutions.

For extracts prepared at 60° C. (FIG. 19B), extracts at concentrations of 1× and above (2×, 4×, 10× and 30×) prepared in ethanol or H₂O₂ are inhibitory for keratinocyte proliferation when used at 1:10 dilution. 4×, 10× and 20× of all extracts including those prepared with H₂O are effective when used at 1:10 dilution. 10× and 30× extracts prepared in H₂O are more effective than those prepared in ethanol or H₂O₂when used at 1:20 and 1:40 dilutions.

Extracts particularly after several rounds of extractions (10× and 30×) prepared in H₂O, ethanol or H₂O₂ at room temperature are inhibitory for keratinocyte proliferation, when used at 1:10 dilution in the MTT assay. 1× and 2× extracts made by H₂O₂ or ethanol are more inhibitory than those made by H₂O when used at 1:10 dilution. 10× and 30× extracts generated by H₂O are more potent than those made by H₂O₂ or ethanol when used at 1:20 and 1:40 dilutions.

No clear differences are observed among concentrated extracts prepared at room temperature (FIG. 19A) versus those prepared at 60° C. (FIG. 19B).

Human primary keratinocytes (NHEK) are sensitive to Spongilla extracts which induce their killing as measured by the MTT assay. Raising the extraction temperature to 60° C. ameliorates the killing of NHEK cells by the extracts. Increasing the concentration of Spongilla material in the extract (up to 25% wt/vol) augments the killing potential. However, 25% is the highest technically possible concentration of Spongilla material in the extract since it becomes very thick and hard to work with at higher concentrations. The order of potency of the solvents used based on the killing potential of the extracts generated is: H₂O₂>EtOH>H₂O for extracts concentrated up to 4× but beyond 10× the order changes to: H₂O>H₂O₂>EtOH.

Furthermore, extracts from lots of Spongilla harvested under specific conditions in the fall (also called “off season”_{—} and processed and stored in accordance with the disclosure herein are bioactive against human primary keratinocytes and that the optimal conditions for maximal activity are to extract 25% (wt/vol) of Spongilla material at 60° C. in H₂O₂ (or in water if at least 10×).

### Example 3

Quality Control of spongilla Harvested for Formulation of Spongilla-Based Therapeutic Compositions for Treating Skin Inflammation

Inflammatory skin disorders covers a broad category that includes many conditions ranging in severity, from occasional rashes accompanied by skin itching and redness, to chronic conditions such as dermatitis (eczema), acne, rosacea, seborrheic dermatitis, psoriasis and others. Skin inflammation can be characterized as acute or chronic. Skin inflammation can be characterized as acute or chronic. Acute inflammation can result from exposure to sun, allergens, or to contact with chemical irritants. This type of inflammation is typically resolved within 1 to 2 weeks with little accompanying tissue destruction. In contrast, chronic inflammation results from a sustained immune cell mediated inflammatory response within the skin itself. This inflammation is long lasting and can cause significant and serious tissue destruction. Inflammatory skin conditions affect over 35 million Americans who annually spend over $2 billion to treat their symptoms.

To demonstrate the anti-inflamatory effect of Spongilla-based therapeutics, 15× Spongilla extracts were prepared in H₂O₂. This extract then was studied in vitro in MCP-1-induced chemotaxis of the human monocytic cell line THP-1 in comparison with 13-Cis-RA.

Cells. THP-1 cells were grown to a confluency of 1×10⁶/ml cells in T75 tissue culture flasks.

Chemotaxis assay. Nuclepore blind well chemotaxis chambers with a lower well volume of 200 μl were used. A maximum volume of 200 μl medium containing RPMI plus 0.1% bovine serum albumin was placed in the lower wells in the presence or absence of various agents. Cells (5×10⁵) were placed in the upper compartments of chemotaxis chambers above the filters. In the lower wells, 10 ng/ml of human recombinant MCP-1 (PeproTech, Inc., NJ) was placed. A dilution of 1:50 or 1:100 of the 15× H₂O₂ extract or 100 nM of 13-Cis-RA was placed in both the upper and lower wells of the chambers. The chambers were incubated for 3 hr at 37° C. in a 5% CO₂ incubator. The filters were then removed, dehydrated, and stained with 15% modified Wright stain for 7 min and then mounted on glass slides using a drop of immersion oil between the filters and the slides. Cells in ten high power fields were counted and averaged for each sample. Migration index was calculated as the number of cells migrating toward the concentration gradients of chemokines, divided by the number of cells migrating toward medium only.

Statistical analysis. Significant values were determined by using the two-tailed Student's t test.

Results

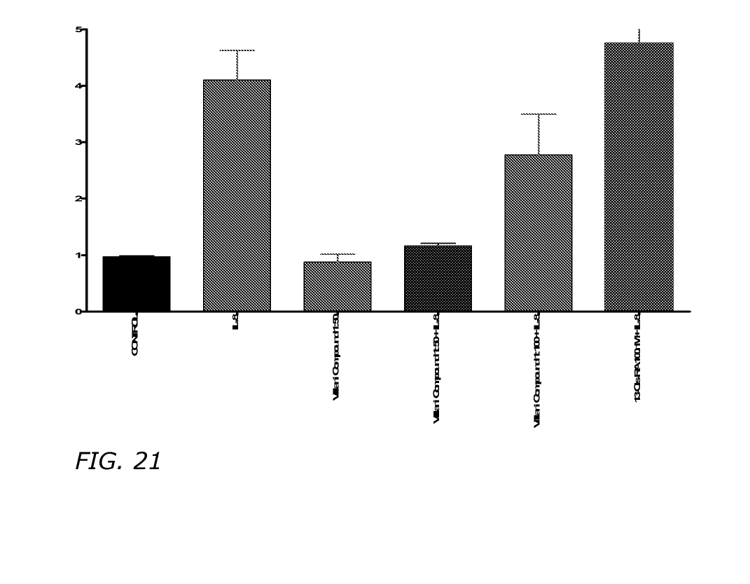

Effect of 15× H₂O₂ extract on the in vitro chemotaxis of THP-1 cells. Modified Boyden chambers to examine the effect of the extract on MCP-1-induced THP-1 chemotaxis. FIG. 20 demonstrates that 10 ng/ml human recombinant MCP-1 significantly induced the chemotaxis of THP-1 cells (P=0.0003 as compared to the control). A dilution of 1:50 extract did not induce the chemotaxis of these cells. However, a 1:50 dilution of the 15× extract significantly inhibited the chemotaxis induced by MCP-1 (P=0.0041 as compared to THP-1 cells migrating in the presence of MCP-1 only). Also, a 1:100 dilution of this extract inhibited MCP-1-induced THP-1 cell chemotaxis (P=0.025 when compared to cells migrating in the presence of MCP-1 alone). 13-Cis-RA had no significant effect on the MCP-induced THP-1 cell migration.

Extract of Spongilla (harvested under specific conditions at summer and processed and stored in accordance with the disclosed methods) prepared in H₂O₂ are a potent inhibitor of the chemotaxis of the monocytic cell line THP-1. Spongilla extracts have potent anti-inflammatory activity, which can be used as a guidance in quality control during the harvest of Spongilla's biometabolites with anti-inflammatory properties.

### Example 5

### Anti-Inflammatory Effects of Spongilla Powder on Interleukin-8-Induced Neutrophil Migration

The aim of this study is to evaluate the anti-inflammatory effects of the 5% H₂O₂ extract of Spongilla powder on interleukin-8-induced neutrophil migration.

Extraction: 1× extracts of Spongilla powder are prepared (at wt/vol 5%) in 5% H₂O₂. The preparations are then added to peripheral human neutrophils and their ability to inhibit interleukin 8-induced migration.

Neutrophil migration assay: Blind-well chemotaxis chambers with a lower-well volume of 200 μl were used. A maximum volume of 200 μl of RPMI medium containing 1% BSA was placed in the lower wells in the presence or absence of IL-8, Spongilla extract, or 13-cis retinoic acid. cells (4×10⁵), Spongilla extract and 13-cis RA were placed in the upper compartments of Boyden chambers above the filters. The chambers were incubated for 45 min at 37° C. in a 5% CO₂ incubator. The filters were then removed, dehydrated, and stained with 15% Giemsa stain for 7 min and then mounted on glass slides. Cells in 10 high-power fields from two filters were counted and averaged for each sample. Migration index was calculated as the number of cells migrating toward the concentration gradient of chemokines divided by the number of cells migrating toward medium only.

Results

The Spongilla extract inhibit IL-8-induced human neutrophil migration in a dose dependent fashion with full inhibition of the migration at a dilution of 1:50 (FIG. 21). 13-CIS RA had no effect on IL-8induced neutrophil migration at 100 nM.

Extract from Spongilla (harvested under certain conditions at summer and processed and stored in accordance with the teachings of the present invention) generated with 5% H₂O₂ is a potent inhibitor of IL-8-induced human neutrophil migration. With these results, the present inventor demonstrated that this extract have potent anti-inflammatory activity, which can be used as a guidance in quality control during the harvest of Spongilla's biometabolites with anti-inflammatory properties.

### Example 6

### Basic Topical Acne Treatment

Certain of the following examples provide formulations using exact amounts in grams and milliliters of each ingredient. However, these exact weights and volumes should not be considered limitations. All of the liquids used herein are aqueous based and contain low percentages of solute. Therefore, the relative weight of each volume of liquid ingredient will be considered equal to the weight of water (1 g/mL). The appended claims will therefore be expressed as ratios. For example, a composition made in accordance with the present disclosure may contain 1.5 g of substantially pure Spongilla powder, 0.5 mL hydrogen peroxide, 2 mL of 5% boric acid, 1 gram of green sea weed powder and 10 mL of floral water. This composition would then be claimed as follows: 1.5 parts of substantially pure Spongilla powder, 0.5 parts 3% hydrogen peroxide, 2 parts of 5% boric acid, 1 part of green sea weed powder and 10 parts of floral water; etc. In certain embodiments, the Spongilla powder is powder of Spongilla lacustris prepared according to the methods above.

Therapeutic compositions prepared from the dried Spongilla powder can be prepared using formulating excipients and procedures known to those skilled in the art of topical medicament preparation. For example, in one embodiment of the present invention topical acne therapeutic comprises of 1.0 grams of Spongilla powder (active ingredient) and 2.0 milliliter of 3% hydrogen peroxide (vehicle). The ingredients are combined and mixed together. The mixture then is heated in the microwave for about 7 seconds. During heating hydrogen peroxide transforms into water and oxygen, which results in fluffy mask of homogeneous consistency. The therapeutic is then applied to the entire face in massaging circular motions, left on for 15 to 30 minutes, and then washed off with water. Recommended usage is every 5 to 7 days.

### Example 7

### Topical Acne Treatment for Professional Use

In another embodiment disclosed herein, topical acne therapeutic comprises of 1.5 grams of Spongilla powder, 1.0 milliliter of 3% hydrogen peroxide, and 3.0 milliliters of 5% boric acid. The mixture and application of this topical anti-acne composition for professional use includes mixing the powder with warm liquids just before use. The therapeutic is then applied to the face or other affected area in circular motions, left on for 25 to 30 minutes, and then washed off with water. Recommended usage is every 4 to 5 days.

### Example 8

### Topical Acne Composition for Home Use

In one embodiment disclosed herein, topical acne therapeutic for home use comprises 0.8 grams of Spongilla powder, 0.2 grams of Plantain powder and 2.5 milliliters of enzyme gel. The mixture and application of this topical anti-acne composition for home use includes mixing the powder with liquid just before use. The contents are then applied to the face or other affected area with a brush, left to dry for 15 minutes and then washed off with water. Recommended usage is every day for a week or until face is cleared and then once a week for maintenance.

### Example 9

### Professional Skin Resurfacing Composition

Formulas suitable for professional skin resurfacing comprise of 1.5 grams of Spongilla powder, 0.2 grams of green seaweed powder, 5.0 milliliters of 3% hydrogen peroxide. The mixture and application of this professional skin resurfacing formula includes mixing the powder with hot liquid just before use. The contents are then applied to the face or other affected area in a circular motion for approximately 5 minutes. Mixture is than left to dry for 25 to 30 minutes and then washed off with water. Recommended usage is once a week.

### Example 10

### Topical Oily Skin Treatment

Formulas for treatment of oily skin may comprise 1.0 grams of Spongilla powder and 2.0 milliliters of chamomile, menthol or calendula water. The mixture and application of this professional skin resurfacing formula includes mixing the powder with warm liquids just before use. The contents are left on for 5 to 10 minutes and then washed off with water. Recommended usage is every 2 days until sebum production is suppressed and then every 10 days for maintenance.

### Example 11

### Topical Deep Peeling Skin Treatment

Formulas for deep peeling of the skin may comprise of 1.5 grams of Spongilla powder, 0.3 grams of green seaweed powder, and 5.0 milliliters of 4% hydrogen peroxide. The mixture and application of this professional deep peeling formula includes a 7 day process. On the first day, the face or effected area is to be steamed. The powder is mixed with hot liquid just before use. The contents are then applied to the face or other affected area in a circular motion. After 5 to 6 minutes, a mask is saturated with hydrogen peroxide in circular motions. It is left to dry for 20 minutes and washed off with water. On day 2, the face or affected area is washed with 2% salicylic acid. The contents are then applied to the face or other affected area in a circular motion. After 5 to 6 minutes, a mask is saturated with hydrogen peroxide in circular motions. It is left to dry for 20 minutes and washed off with water. On the third and fourth days boric ointment is applied to effected areas. On the fifth and sixth days, a moisturizer and soothing mask is applied. On the seventh and last day of treatment, the affected area is exfoliated with a scrub. Recommended usage is once a month.

### Example 12

### Treatment of Hyperpigmentation Disorders

Formulas for hyperpigmented spots removal (including but not limited to melasma, age spots, sun-damage, etc.) of the skin may comprise of 1.0 grams of Spongilla powder, 0.2 grams of white seaweed powder and 3.0 milliliters of enzyme gel. The contents are then mixed together and applied to the face or other affected area in massaging circular motions for approximately 10 minutes, left to dry for 25 to 30 minutes and then washed off with water. Recommended usage is twice a week.

### Example 13

### Treatment for Photo-Damaged and Aging Skin

Formulas for photo-damaged skin may comprise of 2.0 grams of Spongilla powder and 5.0 milliliters of jojoba oil. The mixture and application of this formula for photo-damaged and aging skin includes mixing the powder with hot oil just before use. The contents are then massaging into the face or other affected in circular motions for 30 to 45 minutes, left to stay for 25 to 30 minutes and then washed off with water. Recommended usage is once a week.

### Example 14

### Treatment for Seborrhoeic Dermatitis of the Scalp

Formulas for seborrhoeic dermatitis of the scalp comprise 5.0 grams of Spongilla powder, 5.0 milliliters of 3% of hydrogen peroxide and 5.0 milliliters of 2% of boric acid. The mixture and application of this professional skin resurfacing formula includes mixing the powder with hot liquids just before use. The contents are then applied to the affected area in a circular massaging motion. Mixture is than left for 30 minutes and then washed off with water. Recommended usage 5 to 6 days for 8-10 weeks and then, once a month for maintenance.

### Example 15

### Safety Testing

The preceding exemplary embodiments are not intended as limitations and the Porifera compositions may be formulated in myriad ways and still be considered within the scope of the present disclosure. Desiccated Spongilla powdered comprises numerous biologically active compounds beneficial to promoting skin health, promoting healing and reducing scarring. These beneficial compounds include, but may not be limited to antibacterial, anti-inflammatory, antiviral and other organic bioactive agents in addition to inorganic compounds such as iodine, bromine, phosphorus and sulfur.

The exact mechanism of action of the Spongilla compositions remains unknown. Moreover, the natural combination of ingredients contribute to a synergistic effect that may be destroyed or significantly reduced by extraction and purification of the aforementioned active ingredients. However, the safety and efficacy of the Spongilla compositions are detailed in the following, non-limiting disclosure.

I. In Vivo Rabbit Tests

The test article, Desiccated Animal Sponge, Batch: San Pin 2.3.2.560-96, was evaluated for primary skin irritation in accordance with the guidelines of the International Organization for Standardization 10933: Biological Evaluation of Medical Devices, Part 10: Tests for Irritation and Sensitization. Two 0.2 g portions of the test article moistened with 5 drops of 0.9% sodium chloride and vehicle control article were topically applied to the skin of each of three rabbits and left in place for 24 hours. The sites were graded for erythema and edema at 1, 24, 48 and 72 hours after removal of the single sample application. Under the conditions of this study, no erythema and no edema were observed on the skin of the rabbits. The Primary Irritation Index for the test article was calculated to be 0.0. The response of the test article was categorized as negligible.

The test article identified below was evaluated for primary skin irritation in accordance with the guidelines of the International Organization for Standardization 10933: Biological Evaluation of Medical Devices, Part 10: Tests for Irritation and Sensitization. The purpose of this study was to determine the potential for a single topical application of the test article to irritate skin of the rabbit.

### Materials

Test Article: Desiccated Animal Sponge
Identification No.: Batch: San Pin 2.3.2.506-96
Stability Testing: Complete and on file with the sponsor (per sponsor)
Expiration Date: April, 2004
Vehicle: 0.9% Sodium Chloride, sterile saline
Storage Conditions: Dry, dark conditions:
Control Article: Four-ply gauze supplied by the test facility, was cut into 25 mm×25 mm sections and moistened with 5 drops of 3% hydrogen peroxide per section.
Preparation: 0.2 gram portion of the test article (weighted by sponsor prior to submission), Desiccated Animal Sponge-Thistle, was moistened with 5 drops of 0.9% sodium chloride. The test article and saline were mixed to form a paste consistency. The test mixture was applied to the animals' skin and allowed to air dry for 20 minutes, then wrapped with 4-ply gauze.
Test System: Male Rabbits (Oryctolagus cuniculus) New Zealand

Experimental Procedure

On the day prior to treatment, the fur on each rabbit's back was clipped with an electric clipper. On the day of treatment, four sites, two on each side of the back and positioned cranially and caudally, were designated on each rabbit. The sites were free of blemishes that could interfere with the interpretation of results.

A 0.2 g portion of the test article was moistened with 5 drops of saline, and applied to each caudal site (two sites per rabbit) approximately 25 mm×25 mm square. The test article mixture was allowed to air dry for 20 minutes prior to wrapping. The control vehicle was similarly applied to the caudal sites. The trunk of each animal was wrapped with an elastic binder to maintain the test patches in position. Animals were returned to their cages after treatment.

After the 24 hour exposure, the binders, tape, and patches were removed. The sites were gently wiped with a gauze sponge dampened with deionized water in an attempt to remove any remaining residue. Dermal observations for erythema and edema were recorded at 1, 24, 48 and 72 hours after patch removal.

The Primary Irritation Index of the test was calculated following test completion for each animal. The erythema and edema scores obtained at the 24, 48 and 72 hour intervals were added together and divided by the total number of observations. This calculation was conducted separately for the test and control article for each animal. The score for the control was subracted from the score for the test article to obtain the Primary Irritation Score. The Primary Irritation Score (Table 8) for each rabbit was added together and divided by the number of rabbits to obtain the Primary Irritation Index (Table 6).

Results

No irritation was observed on the skin of the rabbits as summarized in Table 6. The Maximum Irritation Response was not applicable. The Primary Irritation Index of the test article was calculated to be 0.0. The irritation calculations are shown below based on the scale in Table 7:

TABLE 6

| | | | | Combined | | |
|---|---|---|---|---|---|---|
| | | | Individual | Primary | Primary | |
| | | | Primary | Irritation | Irritation | |
| Rabbit | Test Score | Control Score | Irritation | Score | Index | Response |
| Number | Average | Average | Score | (CPIS) | (CPIS ÷ 3) | Category |
| 65977 | 0.0 | 0.0 | 0.0 | 0.0 | 0.0 | Negligible |
| 65976 | 0.0 | 0.0 | 0.0 | | | |
| 65975 | 0.0 | 0.0 | 0.0 | | | |

TABLE 7
Classification System for Skin Reaction

| | NUMERICAL |
|---|---|
| Reaction | GRADING |
| Erythema and Eschar Formation | |
| No erythema | 0 |
| Very slight erythema (barely perceptible) | 1 |
| Well-defined erythema | 2 |
| Moderate erythema | 3 |
| Severe erythema (beet redness) to eschar | 4 |
| formation preventing grading of erythema | |
| Edema Formation | |
| No edema | 0 |
| Very slight edema (barely perceptible) | 1 |
| Well-defined edema (edges of area well- | 2 |
| defined by definite raising) | |
| Moderate edema (raised approximately 1 mm) | 3 |
| Severe edema (raised more than 1 mm and | 4 |
| extending beyond exposure area) | |
| Total possible score for irritation | 8 |

NOTE:
Other adverse changes at the skin sites shall be recorded and reported

TABLE 8
Irritation Response Categories in the Rabbit

| RESPONSE | |
|---|---|
| CATEGORY | MEAN SCORE |
| Negligible | 0.0 to 0.4 |
| Slight | 0.5 to 1.9 |
| Moderate | 2.0 to 4.9 |
| Severe | 5.0 to 8.0 |

Under the conditions of this study, no erythema and no edema were observed on the skin of the rabbits. The Primary Irritation Index for the test article was calculated to be 0.0. The response of the test article was categorized as negligible.

II. In Vivo Guinea Pig Tests

The test article described below was evaluated for the potential to cause delayed dermal contact sensitization following repeated occlusive patching in the guinea pig. The study was conducted based on the requirements of the International Organization for Standardization 10993: Biological Evaluation of Medical Devices, Part 10: Tests for Irritation and Sensitization. The susceptibility of the Hartley guinea pig strain to a known sensitizing agent, l-chloro-2,4-dinitrobenzene (DNCB), has been substantiated.

The study was conducted in accordance with the provisions of the FDA Good Laboratory Practice (GLP) Regulations, 21 CFR 58

### Materials

Test Article: Desiccated Animal Sponge
Identification No: Batch: San Pin 2.3.2.560-96
Stability Testing: Complete and on file with the sponsor (per sponsor)
Expiration Date: April, 2004
Storage Conditions: Dry, dark conditions
Control Article: Approximate 25 mm×25 mm sections of 4-ply gauze were used as the vehicle.
Preparation: A 0.2 gram portion of the test article (weighed by sponsor prior to submission), Desiccated Animal Sponge, was moistened with 5 drops of 3% hydrogen peroxide. The test article and hydrogen peroxide were mixed to form a paste consistency. The 0.2 grain portion of test article mixture was used for approximately 5 patches applied to the animals' skin. The test mixture was allowed to air dry for 20 minutes, then wrapped with 4-ply gauze.
Species: Female Guinea pig (Cavia porcellus) Crl:(HA) Charles River Laboratories Body Weight Range: 311 grams to 366 grams the day prior to first treatment

The Hartley albino guinea pig has been used historically for sensitization studies. Repeated patching of the test material to fur-clipped intact skin will be employed. Topical applications are related to the human exposure route and will permit the evaluation of dermal contact and/or absorption of potential sensitizers during induction and challenge phases. Reactions directly under the topical application site can be observed. The susceptibility of the Hartley strain to a known sensitizing agent, 1-chloro-2,4-dinitrobenzene (DNCB), has been substantiated.

Experimental Procedure

On the day prior to the first induction treatment, each animal was weighed and identified. The hair was removed with an electric clipper from the left flank of 10 guinea pigs designated as test animals and 5 guinea pigs designated as control animals. Each animal was observed daily for general health.

The following day, an aliquot of the test mixture was applied to an approximate 25 mm×25 mm area of the appropriate animals. The test mixture was allowed to dry for 20 minutes before wrapping. The patch was then secured with hypoallergenic tape to the intact skin. To maintain the occluded patch in position, the trunk of each guinea pig was wrapped with an elastic band.

At 6 to 8 hours, the wraps and patches were removed. The sites were wiped with dry gauze after patch removal to remove any material residue from the skin. Observations for dermal responses were recorded 24 hours following the completion of each test article exposure. Prior to scoring, the sites were wiped with 35% isopropyl alcohol saturated gauze.

The application procedure was repeated three times each week (e.g. Monday-Wednesday-Friday) for 3 weeks until nine applications were made to the left flank of the animals. The hair was clipped the day prior to each application to provide a clear site.

At 13 days after the final induction patch, the hair of each guinea pig was removed with an electric clipper from the right flank area. On the following day, an approximate 25 mm×25 mm section of both the control and test article was applied to the intact skin on the dorsal and ventral regions of the right flank of each test and control guinea pig. The trunk of each guinea pig was wrapped with an elastic band to hold the occluded patch in place.

All wraps and patches were removed 6 to 8 hours later. The sites were wiped with dry gauze after patch removal. At 24 hours after patch removal, the challenged sites and surrounding area were shaved. Observations for dermal reactions were conducted at 2-4 hours following the shave and at 48 and 72 hours after challenge patch removal. Sites were wiped with 35% isopropyl alcohol saturated gauze before scoring at each interval. Evaluations for both the induction and challenge phases were based on dermal reactions which were scored as outlined below in Table 9.

TABLE 9
Dermal Reactions

| ERYTHEMA (ER) | | EDEMA (ED) | |
|---|---|---|---|
| | Numerical | | Numerical |
| Reaction | Grading | Reaction | Grading |
| No erythema | 0 | No edema | 0 |
| Slight erythema | 1 | Slight edema | 1 |
| Well-defined erythema | 2 | Well-defined edema | 2 |
| Moderate erythema | 3 | Moderate edema | 3 |
| Severe erythema to slight | 4 | Severe edema | 4 |
| eschar formation | | | |

Following the challenge patch, any test animal exhibiting a dermal reaction greater than that observed in the challenge control conditions was considered as showing delayed contact sensitization to the test article. Pattern and duration of reactions was also considered in the final evaluation.

Results

Clinical Observations: Individual body weights are presented in Table 10. All animals appeared clinically normal throughout the study.

TABLE 10
Individual Body Weights and Clinical Observations

| | Individual Observation | |
|---|---|---|
| Animal Number/ | Pretreatment | Clinical |
| Group | Body Weight (g) | Observations |
| 1 Test | 327 | Appeared normal |
| 2 Test | 354 | Appeared normal |
| 3 Test | 325 | Appeared normal |
| 4 Test | 311 | Appeared normal |
| 5 Test | 344 | Appeared normal |
| 6 Test | 333 | Appeared normal |
| 7 Test | 324 | Appeared normal |
| 8 Test | 333 | Appeared normal |
| 9 Test | 343 | Appeared normal |
| 10 Test | 334 | Appeared normal |
| 11 Control | 366 | Appeared normal |
| 12 Control | 347 | Appeared normal |
| 13 Control | 325 | Appeared normal |
| 14 Control | 355 | Appeared normal |
| 15 Control | 329 | Appeared normal |

Dermal Observations: Individual results of dermal scoring for the induction and challenge phases appear in Tables 11 and 12. No evidence of sensitization was observed. All procedures were conducted in conformance with good laboratory practice and ISO 17025.

Under the conditions of this study, the Spongilla therapeutic compositions did not show any evidence of delayed dermal contact sensitization in the guinea pig. Thus, when Spongilla preparations are prepared in accordance with the teachings of the present invention there are no demonstrable toxic and allergic reactions induced in the recipient.

TABLE 11
Guinea Pig Sensitization Dermal Reactions - Induction

| Animal | Induction Patch Number | | | | | | | | | | | | | | | | | |
|---|---|---|---|---|---|---|---|---|---|---|---|---|---|---|---|---|---|---|
| Number/ | 1 | | 2 | | 3 | | 4 | | 5 | | 6 | | 7 | | 8 | | 9 | |
| Group | ER | ED | ER | ED | ER | ED | ER | ED | ER | ED | ER | ED | ER | ED | ER | ED | ER | ED |
| Test | 0 | 0 | 0 | 0 | 0 | 0 | 0 | 0 | 0 | 0 | 0 | 0 | 0 | 0 | 0 | 0 | 0 | 0 |
| 2 Test | 0 | 0 | 0 | 0 | 0 | 0 | 0 | 0 | 0 | 0 | 0 | 0 | 0 | 0 | 0 | 0 | 0 | 0 |
| 3 Test | 0 | 01 | 0 | 0 | 0 | 0 | 0 | 0 | 0 | 0 | 0 | 0 | 0 | 0 | 0 | 0 | 0 | 0 |
| 4 Test | 0 | 0 | 0 | 0 | 0 | 0 | 0 | 0 | 0 | 0 | 0 | 0 | 0 | 0 | 0 | 0 | 0 | 0 |
| 5 Test | 0 | 0 | 0 | 0 | 0 | 0 | 0 | 0 | 0 | 0 | 0 | 0 | 0 | 0 | 0 | 0 | 0 | 0 |
| 6 Test | 0 | 0 | 0 | 0 | 0 | 0 | 0 | 0 | 0 | 0 | 0 | 0 | 0 | 0 | 0 | 0 | 0 | 0 |
| 7 Test | 0 | 0 | 0 | 0 | 0 | 0 | 0 | 0 | 0 | 0 | 0 | 0 | 0 | 0 | 0 | 0 | 0 | 0 |
| 8 Test | 0 | 0 | 0 | 0 | 0 | 0 | 0 | 0 | 0 | 0 | 0 | 0 | 0 | 0 | 0 | 0 | 0 | 0 |
| 9 Test | 0 | 0 | 0 | 0 | 0 | 0 | 0 | 0 | 0 | 0 | 0 | 0 | 0 | 0 | 0 | 0 | 0 | 0 |
| 10 Test | 0 | 0 | 0 | 0 | 0 | 0 | 0 | 0 | 0 | 0 | 0 | 0 | 0 | 0 | 0 | 0 | 0 | 0 |
| 11 control | 0 | 0 | 0 | 0 | 0 | 0 | 0 | 0 | 0 | 0 | 0 | 0 | 0 | 0 | 0 | 0 | 0 | 0 |
| 12 Control | 0 | 0 | 0 | 0 | 0 | 0 | 0 | 0 | 0 | 0 | 0 | 0 | 0 | 0 | 0 | 0 | 0 | 0 |
| 13 Control | 0 | 0 | 0 | 0 | 0 | 0 | 0 | 0 | 0 | 0 | 0 | 0 | 0 | 0 | 0 | 0 | 0 | 0 |
| 14 Control | 0 | 0 | 0 | 0 | 0 | 0 | 0 | 0 | 0 | 0 | 0 | 0 | 0 | 0 | 0 | 0 | 0 | 0 |
| 15 Control | 0 | 0 | 0 | 0 | 0 | 0 | 0 | 0 | 0 | 0 | 0 | 0 | 0 | 0 | 0 | 0 | 0 | 0 |

Test = Test article
Control = Control article
ER = Erythema
ED = Edema

TABLE 12
Dermal Reactions - Challenge

| | Hours After Patch Removal | | | | | | | | | | | |
|---|---|---|---|---|---|---|---|---|---|---|---|---|
| | 24 | | | | 48 | | | | 72 | | | |
| Animal | Test | | Control | | Test | | Control | | Test | | Control | |
| Number/ | Site | | Site | | Site | | Site | | Site | | Site | |
| Group | ER | ED | ER | ED | ER | ED | ER | ED | ER | ED | ER | ED |
| 1 Test | 0 | 0 | 0 | 0 | 0 | 0 | 0 | 0 | 0 | 0 | 0 | 0 |
| 2 Test | 0 | 0 | 0 | 0 | 0 | 0 | 0 | 0 | 0 | 0 | 0 | 0 |
| 3 Test | 0 | 0 | 0 | 0 | 0 | 0 | 0 | 0 | 0 | 0 | 0 | 0 |
| 4 Test | 0 | 0 | 0 | 0 | 0 | 0 | 0 | 0 | 0 | 0 | 0 | 0 |
| 5 Test | 0 | 0 | 0 | 0 | 0 | 0 | 0 | 0 | 0 | 0 | 0 | 0 |
| 6 Test | 0 | 0 | 0 | 0 | 0 | 0 | 0 | 0 | 0 | 0 | 0 | 0 |
| 7 Test | 0 | 0 | 0 | 0 | 0 | 0 | 0 | 0 | 0 | 0 | 0 | 0 |
| 8 Test | 0 | 0 | 0 | 0 | 0 | 0 | 0 | 0 | 0 | 0 | 0 | 0 |
| 9 Test | 0 | 0 | 0 | 0 | 0 | 0 | 0 | 0 | 0 | 0 | 0 | 0 |
| 10 Test | 0 | 0 | 0 | 0 | 0 | 0 | 0 | 0 | 0 | 0 | 0 | 0 |
| 11 Control | 0 | 0 | 0 | 0 | 0 | 0 | 0 | 0 | 0 | 0 | 0 | 0 |
| 12 Control | 0 | 0 | 0 | 0 | 0 | 0 | 0 | 0 | 0 | 0 | 0 | 0 |
| 13 Control | 0 | 0 | 0 | 0 | 0 | 0 | 0 | 0 | 0 | 0 | 0 | 0 |
| 14 Control | 0 | 0 | 0 | 0 | 0 | 0 | 0 | 0 | 0 | 0 | 0 | 0 |
| 15 Control | 0 | 0 | 0 | 0 | 0 | 0 | 0 | 0 | 0 | 0 | 0 | 0 |

ER = Erythema
ED = Edema

### Example 16

### Exemplary Method for Using the Spongilla Compositions

A pre-weighed package containing substantially pure Spongilla powered is provided. The pre-weighed amount is sufficient for one application to the face. Alternatively, this application can be used to treat the chest, neck, or shoulders instead of face. The treatment may be performed by a medical doctor, a nurse or patient, trained in the procedure. It can be safely administered to patients of all skin types.

The patient should be informed to avoid unprotected sun exposure for two weeks prior to each treatment. Products such as glycolic acid (over-the counter-strength), Retin-A, and Renova should be discontinued for at least 14 days prior to treatment. A longer period of time (several months) must be allowed to pass following professional strength glycolic acid, Jessner's, phenol and TCA peels; CO₂ laser resurfacing and Erbium laser peels. However, in medical office each case may be examined individually.

The treatment areas should be washed with a mild cleanser after make-up is removed. The person performing the procedure may wear protective gloves (it is not a requirement).

Measure out two milliliters of 3% hydrogen peroxide into a small (about 30 ml-50 ml) non-metal container. Gently heat in a microwave to bring the solution to a lukewarm temperature. Using a 1300 watt microwave, time setting is usually 2-10 seconds depending on whether the container is glass or plastic. Pour the warmed peroxide solution into the pre-weighed container of Spongilla powder. Gently stir to obtain a thin paste of fairly homogeneous consistency. Alternatively, the powder can be mixed with the hydrogen peroxide first and then warmed.

Apply one quarter of the amount to the mid-forehead by massaging in circular motions. Spread the paste gently with your fingertips in small circular movements up to the hairline using even pressure. As this paste begins to dry, repeat the same steps as you move down the temples, cheeks, nose and chin. Mild to moderate erythema usually develops. A sensation of sharp crystal needles under the skin is commonly experienced during application. It is best to avoid the periorbital area because the skin is thinner and more sensitive. It is extremely important to massage the paste into the skin before it dries to optimize penetration of active ingredients into the skin. This may take 5 minutes for the face. The neck and chest should always be done last. The most comfortable way to rinse the face is to have the patient splash cold water rather than to wipe their face with a wet washcloth. The skin erythema and tingling sensation of the skin gradually fades over the following 12-24 hours.

Recommended Treatment Times*

| | Total Suggested Time |
|---|---|
| Face | 10-30 minutes |
| Neck | 5-20 minutes |
| Chest | 5-20 minutes |

*Treatment times may vary. Stop immediately if the patient experiences pain and discomfort. Wash the skin with cool water immediately.

Instruct the patient to use a sunscreen with SPF 30-40, preferably one with zinc to help soothe the skin, for at least 2 weeks after each treatment. Explain that failure to do so may result in hyperpigmentation. For the best results, the patient should avoid applying a moisturizer for at least 12 hours. Rich moisturizers should be avoided during the course of treatment. Use of powder make-up or a light foundation can be resumed after the first 12 hours. All patients must be instructed to do the exfoliating on the 5^{th}-6^{th} day at home with scrub provided or return to the office for exfoliating procedure.

In one embodiment a patient was treated using the therapeutic compositions prepared from the dried Spongilla powder and formulated as disclosed above. More specifically, the topical therapeutic was formulated as a topical acne therapeutic comprising of 1.0 grams of Spongilla powder and 2.0 milliliter of 3% hydrogen peroxide mixed prior to use and heated in the microwave for 7 seconds. The topical composition was applied to the entire face with circular motions under the supervision of a trained physician. The treatment was left in contact with the patient's skin for 25 minutes, and then washed off with water. This treatment protocol was repeated every 7 days for 4 weeks.

The therapeutic compositions are derived from Porifera species and can be used to treat myriad skin diseases and disorders. Specifically provided herein are Spongilla-derived topical therapeutics effective in the treatment of acne vulgaris, rosacea, seborrheic dermatitis, eczema (atopic dermatitis), psoriasis, photo-aging, actinic keratosis, and great number of other bacterial, viral, and fungal diseases as well as skin pigmentation disorders, wound healing, and the reduction in the appearance of scars. Several exemplary embodiments are provided; however, it is understood by those skilled in the art of pharmaceutical compounding that many other compositions are possible without departing from the spirit of the claimed invention.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A method of preparing a Spongilla powder for the preparation of a therapeutic composition for the treatment of skin conditions, the method comprising:
harvesting fresh-water sponges of the species Spongilla lacustris from a body of fresh water in the Astrakhan region of Russia during a harvest period;
removing gross contamination from the harvested S. lacustris;
washing the harvested S. lacustris;
drying the harvested S. lacustris for a period of time not to exceed about 21 days from the day of harvest;
grinding and sieving of the dried S. lacustris to form a particle size less than about 2 mm and repeating the grinding and sieving process to form a substantially pure Spongilla powder having an average particle size of less than about 0.2 mm; and
analyzing the resulting substantially pure Spongilla powder to provide a Certificate of Analysis for each lot of Spongilla powder describing the lot's composition, purity and activity to yield reproducible lots of substantially pure Spongilla powder suitable for clinical use.

2. The method of claim 1, further comprising storing the dried S. lacustris material before grinding.

3. The method of claim 1, wherein the harvest period is the summer of each year.

4. The method of claim 1, wherein the harvest period is the fall of each year.

5. The method of claim 1, wherein the harvest is halted if the environmental conditions change more than about 20% during the harvest period.

6. The method of claim 1, wherein the harvested S. lacustris is dried outdoors.

7. The method of claim 1, wherein the harvested S. lacustris is dried near the harvest location.

8. The method of claim 1, wherein the harvested S. lacustris is dried at an ambient temperature of more than about 60° F.

9. The method of claim 1, wherein the harvested S. lacustris is dried at an ambient relative humidity of below about 90%.

10. The method of claim 1, wherein the harvested S. lacustris is dried for at least about 14 days.

11. The method of claim 10, wherein the harvested S. lacustris is dried for at least about 18 days.

12. The method of claim 11, wherein the harvested S. lacustris is dried for about 21 days.

13. The method claim 1, wherein the dried S. lacustris has a residual moisture content of about 0.1% to about 10%.

14. The method of claim 13, wherein the dried S. lacustris has a residual moisture content of about 0.1% to about 5%.

15. The method of claim 14, wherein the dried S. lacustris has a residual moisture content of about 0.1% to about 2%.

16. The method of claim 1, wherein the sieving step comprises passing the ground S. lacustris through a series of sieves with progressively smaller apertures wherein the last aperture is about 0.2 mm.

17. The method of claim 1, wherein the skin condition is acne vulgaris, rosacea, seborrheic dermatitis, atopic dermatitis, psoriasis, photo-aging, or actinic keratosis.

18. The method of claim 1, wherein the skin condition is wound healing or reducing the appearance of scars.

19. A method of preparing a Spongilla powder for the preparation of a therapeutic composition for the treatment of skin conditions, the method comprising:
(a) sampling fresh-water sponges of the species Spongilla lacustris from a body of fresh water in the Astrakhan region of Russia during a potential harvest period;
(b) determining a bioactivity profile of the S. lacustris sampled in step (a) to establish a harvest period;
(c) recording the environmental conditions at the harvest location during the time period established in step (b), wherein the environmental conditions are air temperature, water temperature, relative humidity, precipitation, wind speed, salinity, and oxygenation of the body of fresh water at the harvest location;
(d) harvesting the S. lacustris during the harvest period established in step (b);
(e) removing gross contamination from the harvested S. lacustris;
(f) washing the harvested S. lacustris;
(g) drying the harvested S. lacustris for a period of time not to exceed about 21 days from the day of harvest;
(h) grinding and sieving of the dried S. lacustris to form a particle size less than 2 mm and repeating the grinding and sieving process to form a substantially pure Spongilla powder having an average particle size of less than about 0.2 mm; and
(i) analyzing the resulting substantially pure Spongilla powder to provide a Certificate of Analysis for each lot of Spongilla powder describing the lot's composition, purity and activity to yield reproducible lots of substantially pure Spongilla powder suitable for clinical use.

20. The method of claim 19, wherein determining the bioactivity profile comprises performing high-performance liquid chromatography and at least one assay of a sebocyte proliferation assay, a keratinocyte proliferation assay, and an anti-inflammation assay on the sample, and wherein the harvest period is established if the bioactivity profile of the sample is substantially equivalent to a previously established preferred bioactivity profile.
